(19) United States
(12) Patent Application Publication
(10) Pub. No.: US 2023/0364084 A1
(43) Pub. Date: Nov. 16, 2023

(54) METHODS FOR PREVENTING OR TREATING CONDITIONS RELATED TO PIKFYVE ACTIVITY

(71) Applicant: The Regents of the University of Michigan, Ann Arbor, MI (US)

(72) Inventors: Yuanyuan Qiao, Ann Arbor, MI (US); Arul Chinnaiyan, Ann Arbor, MI (US)

(21) Appl. No.: 18/033,747

(22) PCT Filed: Oct. 28, 2021
(86) PCT No.: PCT/US2021/057022

§ 371 Date: Apr. 25, 2023

Related U.S. Application Data
(60) Provisional application No. 63/106,704, filed on Oct. 28, 2020.

(51) Int. Cl. A61K 31/506; A61K 39/395; A61P 35/00

(52) U.S. Cl. CPC A61K 31/506; A61K 39/3955; A61P 35/00

(57) ABSTRACT

Provided herein are compositions and methods for preventing, attenuating or treating disorders characterized with characterized with PIKfyve-expressing cells. In particular, provided herein are methods for preventing, attenuating or treating disorders characterized with PIKfyve-expressing cells through use of compositions comprising a therapeutic agent capable of inhibiting PIKfyve activity.

### CROSS REFERENCE TO RELATED APPLICATION

This application claims benefit of priority to U.S. Provisional Application No. 63/106,704, filed Oct. 28, 2020, the contents of which are incorporated herein by reference in their entirety.

### FIELD OF THE INVENTION

Provided herein are compositions and methods for preventing, attenuating, or treating disorders characterized with characterized with PIKfyve-expressing cells. In particular, provided herein are methods for preventing, attenuating, or treating disorders characterized with PIKfyve-expressing cells through use of compositions comprising a therapeutic agent capable of inhibiting PIKfyve activity.

### INTRODUCTION

One in nine men will be diagnosed with prostate cancer in their lifetime, and prostate cancer remains the second leading cause of cancer-related death in men in the United States¹. Although advanced prostate cancer often responds to therapies that suppress androgen signaling, resistance inevitably develops, leading to the emergence of castration-resistant prostate cancer (CRPC)². Several therapeutic advancements in the past decade have redefined the treatment of CRPC, including enzalutamide and abiraterone, agents that target continued androgen receptor (AR) signaling^{3,4}. However, these and other therapies for CRPC are not curative, and novel approaches to treat advanced prostate cancer are urgently needed.

Accordingly, improved methods for treating CRPC are desperately needed.

The present invention addresses this urgent need.

### SUMMARY OF THE INVENTION

In response to the prevailing hypothesis that combination treatments may be required to achieve durable responses in advanced cancers^{5,6}, the utility of multi-tyrosine kinase inhibitors (MTKIs) has been explored in recent years. MTKIs inherently hit multiple targets and thus induce effects similar to those observed from combination regimens. Despite promising phase II clinical trial results with the MTKI cabozantinib in CRPC⁷, a recent phase III trial failed to meet its primary survival endpoint⁸.

Experiments conducted during the course of developing embodiments for the present invention determined whether alternative phase I-cleared MTKIs could be repositioned for treatment of advanced prostate cancer and have identified ESK981 as an effective monotherapy in several preclinical models with uniquely described properties that also potentiate immunotherapeutic responses.

ESK981 (13-isobutyl-4-methyl-10-(pyrimidin-2-ylamino)-1,2,4,7,8,13-hexahydro-6H-indazolo[5,4-a]pyrrolo[3,4-c]carbazol-6-one;

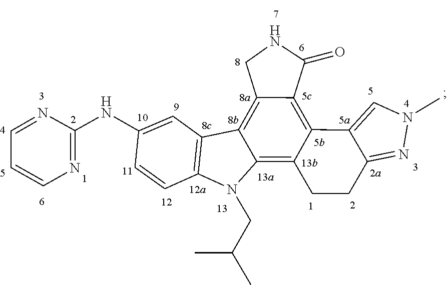 [SMILES: CC(C)Cn1c2ccc(Nc3ncccn3)cc2c2c3c(c4c(c21)CCc1nn(C)cc1-4)C(=O)NC3]

formerly known as CEP-11981) is a novel oral MTKI that was originally developed by Cephalon⁹. ESK981 was initially identified as an angiogenesis inhibitor that targeted several pathways involved in the angiogenic response, but without the off-target activities of other MTKIs (i.e., sunitinib and sorafenib) that result in adverse events¹⁰. ESK981 has potent activity against kinases implicated in angiogenesis, including VEGFR-1 (FLT1), VEGFR-2 (KDR), and Tie-2 (TEK)⁹. Importantly, ESK981 has already cleared a phase I dose-escalation clinical trial, where it demonstrated favorable safety, pharmacokinetic, and pharmacodynamic profiles in patients with advanced, relapsed, or refractory solid tumors¹¹. Furthermore, 51% of evaluable patients achieved stable disease when measured at ≥6 weeks of ESK981 treatment, and this value increased in the highest dosing cohorts.

Serendipitously, the experiments described herein uncovered a novel mechanism of action of ESK981 that involves robust accumulation of autophagosomes and lysosomes through direct inhibition of the lipid kinase PIKfyve. As a class III lipid kinase, PIKfyve converts phosphatidylinositol 3-phosphate (PI(3)P) to phosphatidylinositol 3,5-bisphosphate (PI(3,5)P₂)¹². Studies of PIKfyve as a therapeutic target have been limited to non-Hodgkin's lymphoma, multiple myeloma, and non-small cell lung cancer¹³⁻¹⁵, and its therapeutic potential has not been fully investigated in other solid malignancies. Such experiments identified ESK981 as a novel PIKfyve inhibitor that confers potent tumor inhibition by blocking autophagic flux in advanced prostate cancer.

The role of autophagy has been intensely studied in cancer¹⁶, and its role in immunogenic cell death is emerging^{17,18}. Several reports have suggested that autophagy inhibition may sensitize tumors to immune checkpoint inhibitors through release of T cell attracting chemokines^{19,20} or other immunomodulatory mechanisms, such as restoring surface expression of MHC-I²¹. In this study, we demonstrate that inhibition of autophagic flux, triggered by ESK981 or PIKfyve inhibition, renders prostate cancer cells toward a more immune responsive state, conferring sensitivity to anti-tumor immunotherapy. Experiments described herein indicates that discovery of other compounds like ESK981 that target autophagic flux and/or PIKfyve as an effective strategy to activate anti-tumor immune responses. These findings have important implications for those cancers, such as prostate, that are not often intrinsically immunogenic and have had limited success with immunotherapy^{22,23}.

Accordingly, the present invention provides compositions and methods for preventing, attenuating, or treating disorders characterized with characterized with PIKfyve-expressing cells. In particular, provided herein are methods for preventing, attenuating, or treating disorders characterized with PIKfyve-expressing cells through use of compositions comprising a therapeutic agent capable of inhibiting PIKfyve activity.

The present invention contemplates that agents capable of inhibiting PIKfyve activity satisfy an unmet need for the treatment of multiple cancer types characterized cells having increased PIKfyve activity, either when administered as monotherapy to induce cell growth inhibition, apoptosis and/or cell cycle arrest in cancer cells, or when administered in a temporal relationship with additional agent(s), such as other cell death-inducing or cell cycle disrupting cancer therapeutic drugs (e.g., immune checkpoint inhibitors) or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of an agent capable of inhibiting PIKfyve activity (e.g., ESK981) and a course of an anticancer agent produces a greater tumor response and clinical benefit in such animals compared to those treated with the compound or anticancer drugs/radiation alone. Since the doses for all approved anticancer drugs and radiation treatments are known, the present invention contemplates the various combinations of them with the present compounds.

As noted, the Applicants have found that the compound ESK981 function inhibitors of PIKfyve activity, and serve as therapeutics for the treatment of cancers characterized with increased PIKfyve-expressing cells (e.g., prostate cancer cells characterized with increased PIKfyve activity) and other related diseases.

The embodiments of the present invention are not limited to specific certain agents capable of inhibiting PIKfyve activity. In some embodiments, the agent is any type or kind of moiety (e.g., small molecule, polypeptide or peptide fragment, antibody or fragment thereof, nucleic acid molecule (e.g., RNA, siRNA, microRNA, interference RNA, mRNA, replicon mRNA, RNA-analogues, and DNA), etc.) capable of inhibiting PIKfyve activity. In some embodiments, the agent is any type or kind of moiety (e.g., small molecule, polypeptide or peptide fragment, antibody or fragment thereof, nucleic acid molecule (e.g., RNA, siRNA, microRNA, interference RNA, mRNA, replicon mRNA, RNA-analogues, and DNA), etc.) capable of inhibiting conversion of phosphatidylinositol 3-phosphate (PI(3)P) to phosphatidylinositol 3,5-bisphosphate (PI(3,5)P₂), inhibiting PIKfyve activity related tumor growth, inhibiting PIKfyve activity related autophagic flux, and/or activating an anti-tumor immune response in cells having increased PIKfyve activity.

In some embodiments, the agent is ESK981 or a compound similar to ESK981, or a pharmaceutically acceptable salt, solvate, or prodrug thereof. In some embodiments, the agent is capable of inhibiting conversion of phosphatidylinositol 3-phosphate (PI(3)P) to phosphatidylinositol 3,5-bisphosphate (PI(3,5)P₂). In some embodiments, the agent is capable of inhibiting PIKfyve activity related tumor growth. In some embodiments, the agent is capable of inhibiting PIKfyve activity related autophagic flux. In some embodiments, the agent is capable of activating an anti-tumor immune response in cells having increased PIKfyve activity.

The invention also provides the use of such PIKfyve inhibiting agents to induce cell cycle arrest and/or apoptosis in cells having increased PIKfyve activity (e.g., cancer cells having increased PIKfyve activity). The invention also relates to the use of compounds for sensitizing cells to additional agent(s), such as inducers of apoptosis and/or cell cycle arrest, and chemoprotection of normal cells through the induction of cell cycle arrest prior to treatment with chemotherapeutic agents.

The PIKfyve inhibiting agents are useful for the treatment, amelioration, or prevention of disorders, such as any type of cancer characterized with increased PIKfyve activity (e.g., prostate cancer characterized with PIKfyve-expressing cells).

In certain embodiments, the PIKfyve inhibiting agents can be used to treat, ameliorate, or prevent a cancer characterized with PIKfyve-expressing cells that additionally is characterized by resistance to cancer therapies (e.g., those cancer cells which are chemoresistant, radiation resistant, hormone resistant, and the like). In certain embodiments, the cancer is one or more of prostate cancer, castration resistant prostate cancer, pancreatic cancer, colon cancer, melanoma, lung cancer, breast cancer, renal cancer, lymphoma, ovarian cancer, bladder cancer, Merkel cell carcinoma, rhabdomyosarcoma, osteosarcoma, synovial sarcoma, glioblastoma, Ewing's sarcoma, diffuse intrinsic pontine glioma (DIPG), neuroblastoma, and Wilms' tumor.

In such embodiments, the compounds inhibit the activity of PIKfyve which results in inhibited growth of PIKfyve-expressing cancer cells or supporting cells outright and/or render such cells as a population more susceptible to the cell death-inducing activity of cancer therapeutic drugs (e.g., immune checkpoint inhibitors) or radiation therapies. In some embodiments, the inhibition of PIKfyve-expressing cancer cells activity occurs through, for example, inhibiting conversion of phosphatidylinositol 3-phosphate (PI(3)P) to phosphatidylinositol 3,5-bisphosphate (PI(3,5)P₂), inhibiting PIKfyve activity related tumor growth, inhibiting PIKfyve activity related autophagic flux, and/or activating an anti-tumor immune response in cells having increased PIKfyve activity.

In some embodiments, one or more anticancer agents are co-administered with the PIKfyve inhibiting agent, wherein said anticancer agent one or more of an immune checkpoint inhibitor (e.g., pembrolizumab, nivolumab, cemiplimab, atezolizumab, avelumab, durvalumab, ipilimumab), a chemotherapeutic agent, and radiation therapy.

Such embodiments are not limited to particular type or kind of immune checkpoint inhibitor. In some embodiments, the immune checkpoint inhibitor is selected from a PD-1 inhibitor, PD-L1 inhibitor, CTLA-4 inhibitor, LAG3 inhibitor, TIM3 inhibitor, cd47 inhibitor, TIGIT inhibitor, and B7-H1 inhibitor.

In some embodiments, the PD-1 inhibitor is selected from nivolumab, pembrolizumab, STI-A1014, pidilzumab, and cemiplimab-rwlc.

In some embodiments, the PD-L1 inhibitor is selected from velumab, atezolizumab, durvalumab, and BMS-936559.

In some embodiments, the CTLA-4 inhibitor is selected from ipilimumab and tremelimumab.

In some embodiments, the LAG3 inhibitor is GSK2831781.

The invention also provides pharmaceutical compositions comprising such therapeutic agents capable of inhibiting PIKfyve activity (e.g., ESK981 or compounds structurally similar to ESK981) in a pharmaceutically acceptable carrier.

The invention also provides kits comprising one or more of the described therapeutic agents capable of inhibiting PIKfyve activity (e.g., ESK981 or compounds structurally similar to ESK981) and instructions for administering the compound to an animal. The kits may optionally contain other therapeutic agents, e.g., immune checkpoint inhibitors.

In certain embodiments, the present invention provides methods of treating, ameliorating, or preventing a hyperproliferative disease characterized with PIKfyve-expressing cells in a patient comprising a) obtaining a biological sample from the patient, wherein the biological sample comprises cancer cells associated with a hyperproliferative disease; b) determining the presence or absence of PIKfyve-expression within the cancer cells; c) administering to said patient a therapeutically effective amount of a composition comprising a therapeutic agent capable of inhibiting PIKfyve activity (e.g., ESK981 or compounds structurally similar to ESK981) if the cancer cells are characterized as having PIKfyve-expression. In some embodiments, the hyperproliferative disease is a cancer (e.g., prostate cancer, castration resistant prostate cancer, pancreatic cancer, colon cancer, melanoma, lung cancer, breast cancer, renal cancer, lymphoma, ovarian cancer, bladder cancer, Merkel cell carcinoma, rhabdomyosarcoma, osteosarcoma, synovial sarcoma, glioblastoma, Ewing's sarcoma, diffuse intrinsic pontine glioma (DIPG), neuroblastoma, and Wilms' tumor). In some embodiments, the patient is a human patient. In some embodiments, administration of the agent results in one or more of inhibiting conversion of phosphatidylinositol 3-phosphate (PI(3)P) to phosphatidylinositol 3,5-bisphosphate (PI(3,5)P₂), inhibiting PIKfyve activity related tumor growth, inhibiting PIKfyve activity related autophagic flux, and/or activating an anti-tumor immune response in cells having increased PIKfyve activity. In some embodiments, the methods further comprise administering to said patient one or more anticancer agents, wherein said anticancer agent one or more of an immune checkpoint inhibitor (e.g., pembrolizumab, nivolumab, cemiplimab, atezolizumab, avelumab, durvalumab, ipilimumab), a chemotherapeutic agent, and radiation therapy.

In certain embodiments, the present invention provides methods for inhibiting PIKfyve activity in a subject having PIKfyve-expressing cells through administering to the subject a composition comprising a therapeutically effective amount of an agent capable of inhibiting PIKfyve activity (e.g., ESK981 or a compound similar to ESK981).

In certain embodiments, the present invention provides methods for inhibiting conversion of phosphatidylinositol 3-phosphate (PI(3)P) to phosphatidylinositol 3,5-bisphosphate (PI(3,5)P₂) in a subject having PIKfyve-expressing cells through administering to the subject a composition comprising a therapeutically effective amount of an agent capable of inhibiting PIKfyve activity (e.g., ESK981 or a compound similar to ESK981).

In certain embodiments, the present invention provides methods for inhibiting PIKfyve activity related tumor growth in a subject having PIKfyve-expressing cells (e.g., PIKfyve-expressing cancer cells) through administration to the subject a therapeutically effective amount of an agent capable of inhibiting PIKfyve activity (e.g., ESK981 or a compound similar to ESK981).

In certain embodiments, the present invention provides methods for inhibiting PIKfyve activity related autophagic flux in a subject having PIKfyve-expressing cells through administration to the subject a therapeutically effective amount of an agent capable of inhibiting PIKfyve activity (e.g., ESK981 or a compound similar to ESK981).

In certain embodiments, the present invention provides methods for activating an anti-tumor immune response in a subject having PIKfyve-expressing cells through administration to the subject a therapeutically effective amount of an agent capable of inhibiting PIKfyve activity (e.g., ESK981 or a compound similar to ESK981).

In certain embodiments, the present invention provides methods for inhibiting conversion of phosphatidylinositol 3-phosphate (PI(3)P) to phosphatidylinositol 3,5-bisphosphate (PI(3,5)P₂) in PIKfyve-expressing cells through exposing such cells to compositions comprising an agent capable of inhibiting PIKfyve activity (e.g., ESK981 or a compound similar to ESK981).

In certain embodiments, the present invention provides methods for inhibiting PIKfyve activity in PIKfyve-expressing cells through exposing such cells to compositions comprising an agent capable of inhibiting PIKfyve activity (e.g., ESK981 or a compound similar to ESK981).

In certain embodiments, the present invention provides methods for inhibiting PIKfyve activity related tumor growth in PIKfyve-expressing cells (e.g., PIKfyve-expressing cancer cells) through exposing such cells to compositions comprising an agent capable of inhibiting PIKfyve activity (e.g., ESK981 or a compound similar to ESK981).

In certain embodiments, the present invention provides methods for inhibiting PIKfyve activity related autophagic flux in PIKfyve-expressing cells through exposing such cells to compositions comprising an agent capable of inhibiting PIKfyve activity (e.g., ESK981 or a compound similar to ESK981).

In certain embodiments, the present invention provides methods for activating an anti-tumor immune response in cells having increased PIKfyve activity through exposing such cells to compositions comprising an agent capable of inhibiting PIKfyve activity (e.g., ESK981 or a compound similar to ESK981).

### BRIEF DESCRIPTION OF DRAWINGS

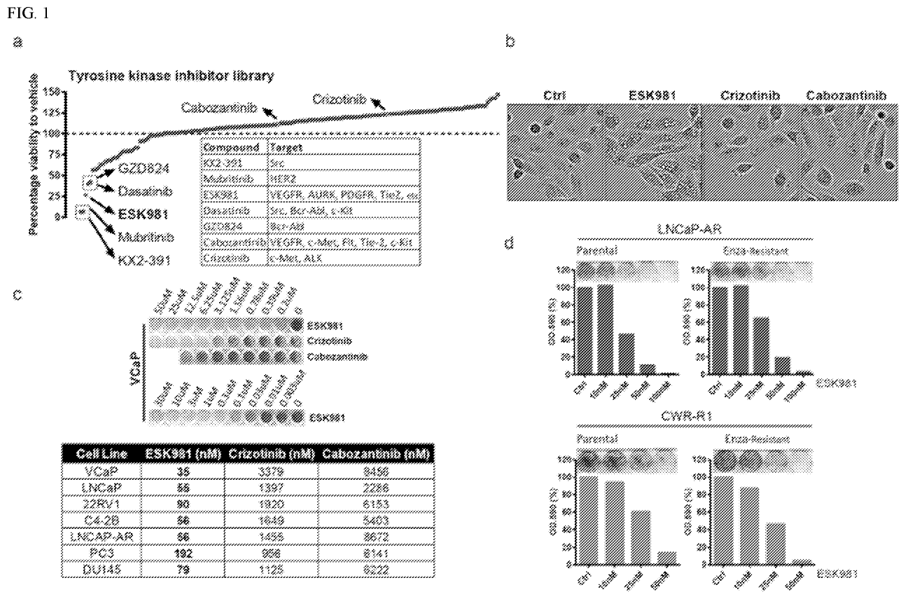

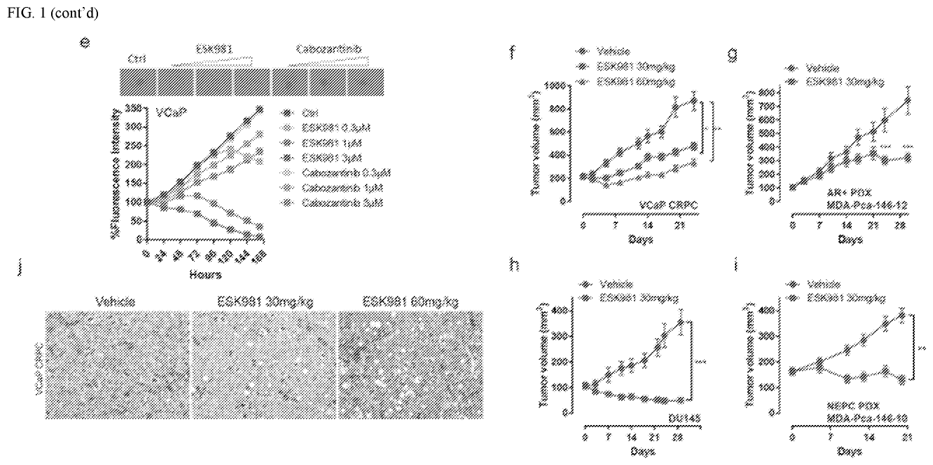

FIG. 1: ESK981 inhibits the growth of diverse preclinical models of prostate cancer and is associated with a unique vacuolization morphology.
(a) The percentage viabilities of DU145 cells treated with 300 nM ESK981 or 167 other tyrosine kinase inhibitors when compared to a vehicle control. The top five most inhibitory compounds, as well as cabozantinib and crizotinib (highlighted in orange), and their respective targets are listed in the table. ESK981 is highlighted in red.
(b) Morphological differences of nuclear-restricted RFP-expressing DU145 cells treated with 300 nM ESK981, crizotinib, or cabozantinib.
(c) A long-term survival assay was used to calculate the half-maximum inhibitory concentration (IC₅₀) after two weeks of incubation with the serial dilutions of indicated drugs. (Top) Long-term survival assays of VCaP prostate cancer cells exposed to MTKIs. (Bottom) IC₅₀ of ESK981, crizotinib, and cabozantinib in a panel of prostate cancer cell lines.
(d) ESK981 was effective against enzalutamide (Enza)-resistant cell lines. LNCaP-AR and CWR-R1 enzalutamide-resistant cells were maintained in 5 μM and 20 μM enzalutamide medium, respectively, in vitro. Long-term survival (two weeks) was assayed by absorbance of crystal violet at OD590.
(e) VCaP-RFP cells were cultured for three days in ultralow attachment plates to form 3D tumor spheroids prior to the indicated drug treatments. Increasing concentrations of ESK981 and cabozantinib were added over the indicated time period. Fluorescence intensity of 3D spheroids was measured by IncuCyte ZOOM.
(f) Castration-resistant VCaP tumors were established subcutaneously in castrated SCID mice, and mice were randomized into three groups, which received vehicle, 30 mg/kg, or 60 mg/kg ESK981 by oral gavage once per day for the indicated dosing schedule. Tumor volumes were monitored by a digital caliper twice per week. Data were analyzed by unpaired t test and presented as mean±SEM. *p<0.05; **p<0.01.
(g) AR⁺ prostate patient-derived xenograft (PDX) MDA-PCa-146-12 were implanted subcutaneously in non-castrated SCID mice, and mice were randomized into two groups receiving either once daily vehicle or 30 mg/kg ESK981 for five days each week. Tumor volumes were taken twice per week by digital caliper. Data were analyzed by unpaired t test and presented as mean±SEM. ***p<0.001.
(h) DU145 tumors were established subcutaneously in non-castrated SCID mice until an average size of 100 mm³, and mice were then randomized into two groups that were treated with vehicle or 30 mg/kg ESK981. Each group received treatment five days per week. Tumor volumes were taken twice per week by digital caliper. Data were analyzed by unpaired t test and presented as mean±SEM. ***p<0.001.
(i) Neuroendocrine (NEPC) prostate patient-derived xenograft MDA-PCa-146-10 were subcutaneously grown into non-castrated SCID mice until tumors reached an average size of 100 mm³, after which mice were randomized into two groups. Mice in each group received either vehicle or 30 mg/kg ESK981 five days per week. Tumor volumes were monitored twice per week. Data were analyzed by unpaired t test and presented as mean±SEM. **p<0.01.
(j) Representative H&E images from castration-resistant VCaP tumors after five days of treatment with vehicle or ESK981 showing a dose-dependent induction of a vacuolization morphology.

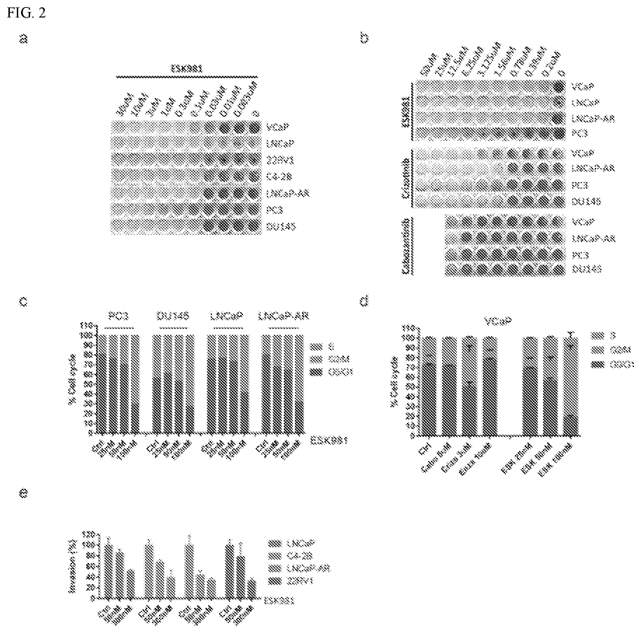

FIG. 2: ESK981 blocks cell growth, induces cell cycle arrest, and decreases cellular invasion.
(a-b) Representative crystal violet staining for a long-term survival assay of a panel of prostate cell lines at various concentrations of ESK981, crizotinib, or cabozantinib.
(c) Cell cycle analysis was measured after 72 hours of increasing concentrations of ESK981 treatment in indicated prostate cancer cell lines. Ctrl, control.
(d) Cell cycle analysis of VCaP cells that were treated with the indicated compounds for 72 hours. Cabo, cabozantinib; Crizo, crizotinib; Enza, enzalutamide; ESK, ESK981.
(e) Matrigel invasion assay of various prostate cancer cell lines that were treated with the indicated concentrations of ESK981. The percentage invasion was quantified with a fluorescent plate reader.

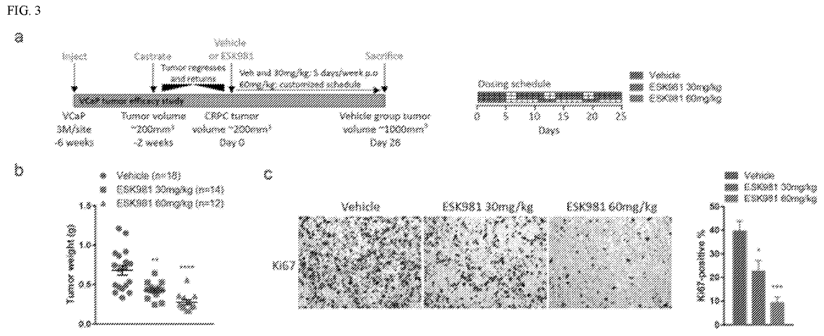

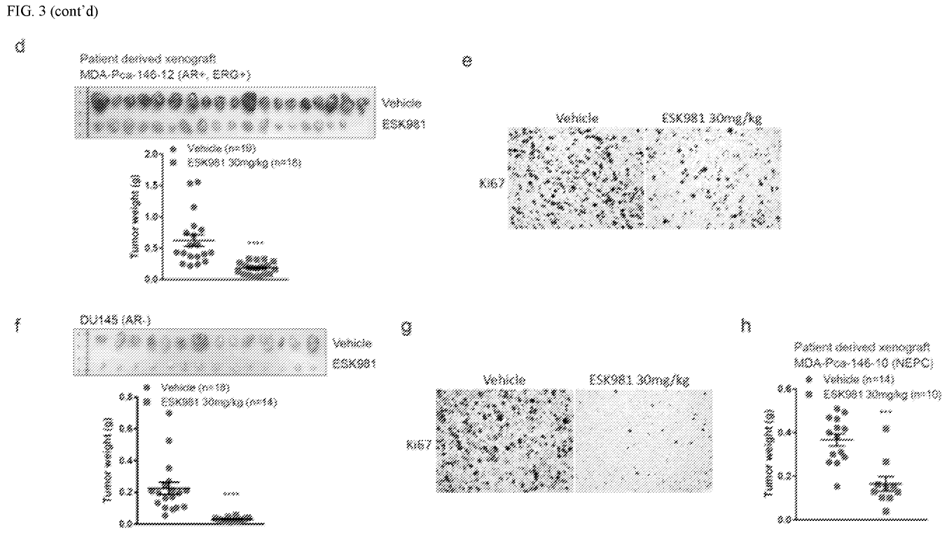

FIG. 3: ESK981 inhibits prostate tumor progression in multiple murine models.
(a) Schematic illustration of the VCaP CRPC mouse xenograft experimental design (left) and dosing schedule (right). p.o, oral gavage.
(b) VCaP tumor weights were measured after complete surgical resection of the tumor from flanks of mice. Data were analyzed by unpaired t test and presented as mean±SEM. **p<0.01; ****p<0.0001.
(c) Representative IHC images for proliferation marker Ki67 are shown after treatment with the indicated drugs for five days in VCaP tumors. Quantification of positive Ki67 percentage is shown on the right. Data were analyzed by unpaired t test and presented as mean±SEM. *p<0.05; ***p<0.001.
(d) AR⁺ and ERG⁺ patient-derived xenograft MDA-PCa-146-12 were established subcutaneously into non-castrated mice until each tumor reached an average size of 100 mm³, after which mice were randomized into two groups that received either vehicle or 30 mg/kg ESK981 five days per week, once per day by oral gavage. (Top) Representative individual tumors from vehicle and ESK981 groups. (Bottom) Tumor weights from individual tumors. Data were analyzed by unpaired t test and presented as mean±SEM. ****p<0.0001.
(e) Representative IHC showing Ki67 staining for vehicle and 30 mg/kg ESK981 groups of MDA-PCa-146-12 tumors.
(f) (Top) Representative individual tumors from vehicle and ESK981 groups of DU145 tumors. (Bottom) DU145 tumor weights were measured after complete surgical resection of the tumor from flanks of mice. Data were analyzed by unpaired t test and presented as mean±SEM. ****p<0.0001.
(g) Representative IHC showing Ki67 staining for the vehicle and 30 mg/kg ESK981 groups of DU145 tumors.
(h) Tumor weight measurement at day 21 for vehicle and 30 mg/kg ESK981 groups of MDA-PCa-146-10 tumors. Data were analyzed by unpaired t test and presented as mean±SEM. ***p<0.001.

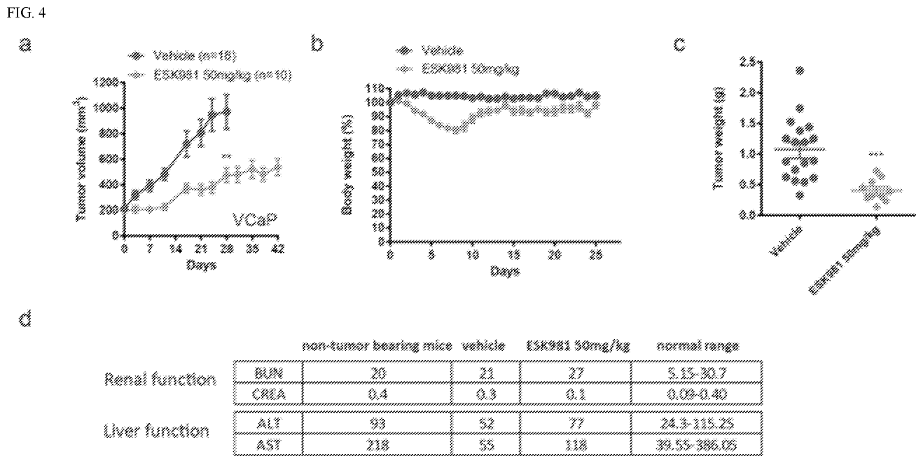

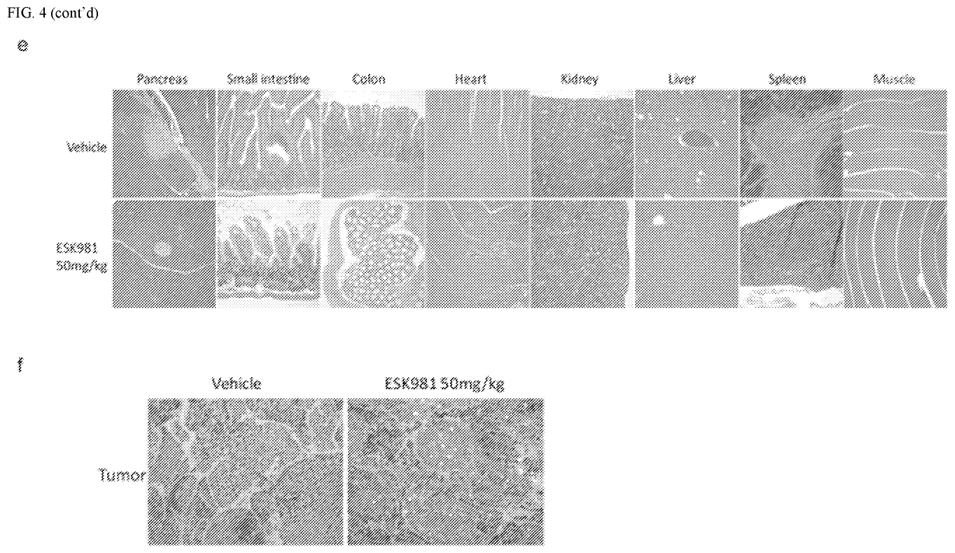

FIG. 4: Renal function, liver function, and histopathological evaluation of ESK981-treated xenografts.
(a) Castration-resistant VCaP tumors were established according to FIG. 3a. Tumor-bearing mice were divided into vehicle and ESK981 50 mg/kg groups, and tumor volumes were monitored twice per week for six weeks. Data were analyzed by unpaired t test and presented as mean±SEM at day 25. **p<0.01.
(b) The body weights of VCaP tumor-bearing mice were monitored daily throughout this study.
(c) The weight of VCaP tumors were measured at the end of this study. Data were analyzed by unpaired t test and presented as mean±SEM. ***p<0.001.
(d) Blood chemistry was evaluated for renal and liver functions in non-tumor-bearing and VCaP tumor-bearing mice in vehicle and 50 mg/kg ESK981 treatment groups.
(e) Representative histological sections showing H&E staining for various organs taken from vehicle- or ESK981-treated mice.
(f) Representative histological sections showing H&E staining for tumors taken from vehicle- or ESK981-treated mice.

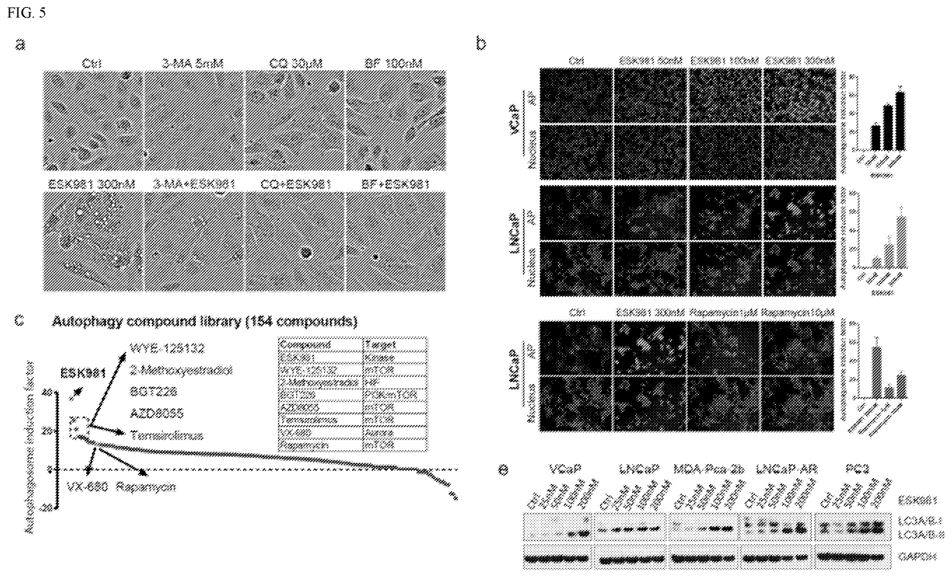

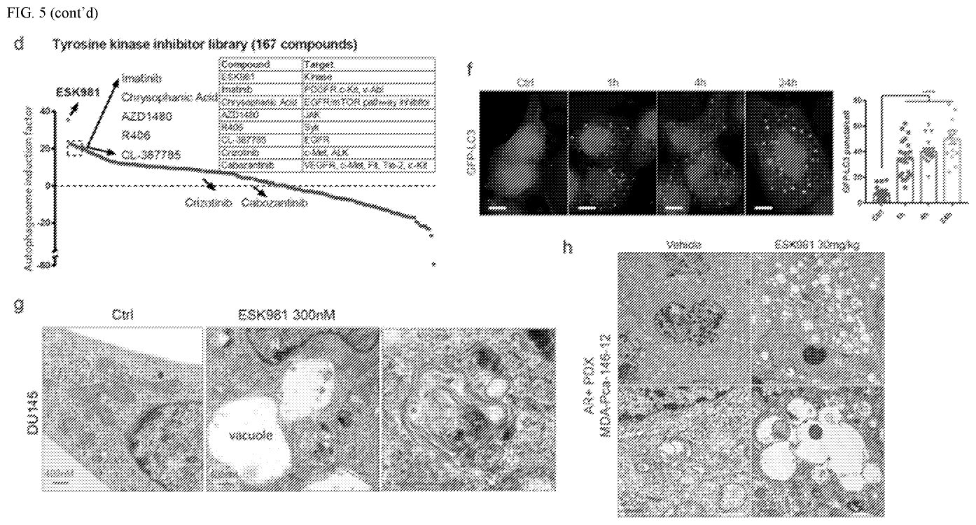

FIG. 5: ESK981 causes accumulation of autophagosomes and lysosomes through inhibition of autophagic flux in prostate cancer cells.
(a) Morphology of DU145-RFP cells treated with either ESK981, autophagy inhibitors (3-methyladenine [3-MA], chloroquine [CQ], bafilomycin A₁ [BF]), or a combination of ESK981 and one additional autophagy inhibitor for six hours. Red indicates nuclei.
(b) VCaP and LNCaP cells were treated with increasing concentrations of ESK981 for 24 hours. Autophagosome induction activity was measured with CYTO-ID®, and the quantification of autophagosomes are shown on the right. Rapamycin served as a positive control for autophagy induction.
(c) Autophagosome induction activity of ESK981, measured with CYTO-ID®, when compared to an autophagy related compound library consisting of 154 compounds. DU145 cells were exposed to 300 nM ESK981 for 24 hours. The top five compounds are presented in the table. VX-680 and rapamycin are highlighted in orange.
(d) Autophagosome induction activity of ESK981, measured with CYTO-ID®, when compared to a tyrosine kinase inhibitor library consisting of 167 compounds. DU145 cells were exposed to 300 nM ESK981 for 24 hours. The top five additional autophagy-inducing compounds, as well as crizotinib and cabozantinib, are presented in the table.
(e) The indicated prostate cancer cell lines were treated with increasing concentrations of ESK981 for 24 hours. LC3 levels were assessed by western blot, with GAPDH serving as a loading control.
(f) Representative images of GFP-LC3 puncta in DU145 cells with 300 nM ESK981 treatment for various times. Scale bar: 10 μm. Quantification of GFP-LC3 puncta is shown on the right. N=20 per group. Data were analyzed by unpaired t test and presented as mean±SEM by GraphPad Prism. ***p<0.001.
(g) TEM micrographs of DU145 cells after 300 nM ESK981 treatment for 24 hours. Micrograph of ESK981-treated cell shows mostly clear vacuoles adjacent to an autophagic vacuole, which is magnified in the red dashed box. Red arrow indicates a mostly clear vacuole. N, nucleus.
(h) Micrographs of MDA-PCa-146-12 PDX tumors taken by TEM after five days of treatment from each group. Red arrows indicate vacuoles in ESK981 group, and yellow arrows indicate cellular materials inside the vacuole. N, nucleus.
(i) Immunofluorescence staining of LAMP1 in DU145 cells treated with control or 300 nM ESK981 for 24 hours.
(j) Lysosomal activity was quantified by FACS after staining with LysoTracker Green. VCaP, LNCaP, PC3, and DU145 cells were treated with increasing concentrations of ESK981 for 24 hours (Left). VCaP, LNCaP, PC3, and DU145 cells were treated with DMSO, ESK981 (300 nM), bafilomycin A₁ (100 nM), or ESK981-bafilomycin A₁ combination for 24 hours (Right).
(k) Ratio of GFP/RFP signal in PC3 and DU145 GFP-LC3-RFP-LC3ΔG stable expressing cells with the indicated treatment for 24 hours. *p<0.05; **p<0.01.
(l) Paired MEF cells with either Atg5 wild type (Atg5^{+/+}) or Atg5 knockout (Atg5^{-/-}) were treated with 300 nM ESK981 for 24 hours. (Left) Morphologies are shown in phase contrast microscopy. (Right) Atg5 and LC3 protein levels were examined by western blot.

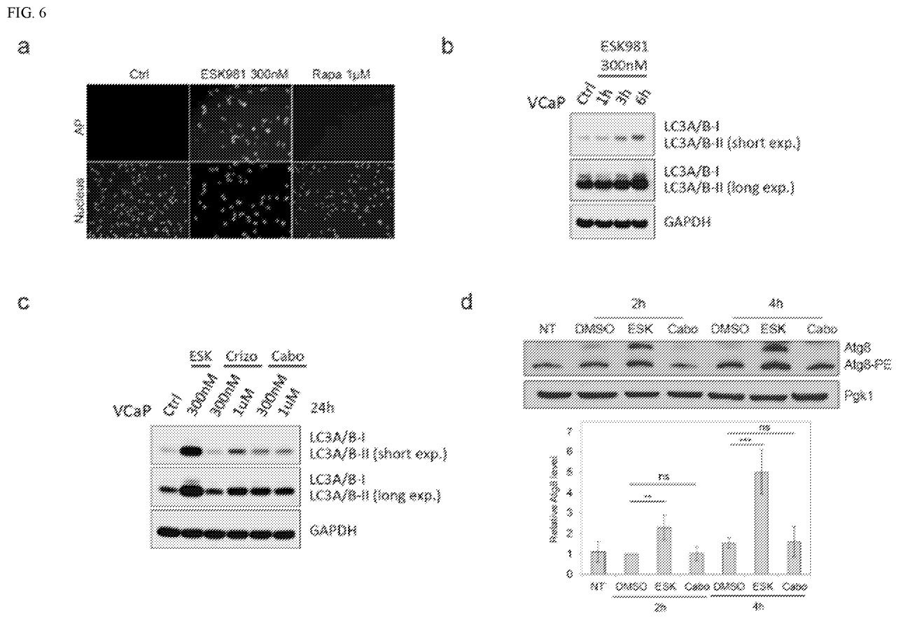

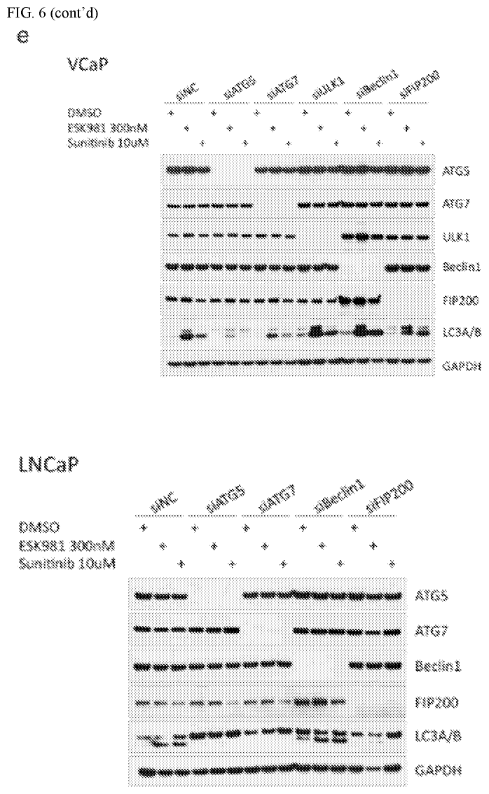

FIG. 6: ESK981 robustly induces autophagosome levels and is dependent on ATG5 for its effects.
(a) DU145 cells with the indicated drug treatment for 24 hours. Autophagosome induction activity was visualized by CYTO-ID® assay. Rapa, rapamycin.
(b) VCaP cells were treated with 300 nM ESK981 for the indicated time points, and LC3 protein levels were assessed by western blot.
(c) VCaP cells were treated with ESK981 (ESK), crizotinib (Crizo), and cabozantinib (Cabo) at the indicated concentrations. Protein levels of LC3 were examined after 24 hours of treatment.
(d) Protein levels of Atg8 in yeast prd5Δ cells after ESK981 (ESK) or cabozantinib (Cabo) treatment under nitrogen deprivation conditions. NT, no treatment. Data were analyzed by unpaired t test and presented as mean±SEM. **p<0.01; ***p<0.001.
(e) Protein levels of indicated protein post various siRNA knockdown in VCaP and LNCaP cells with or without 300 nM ESK981 or 1 μM sunitinib treatment for 24 hours.

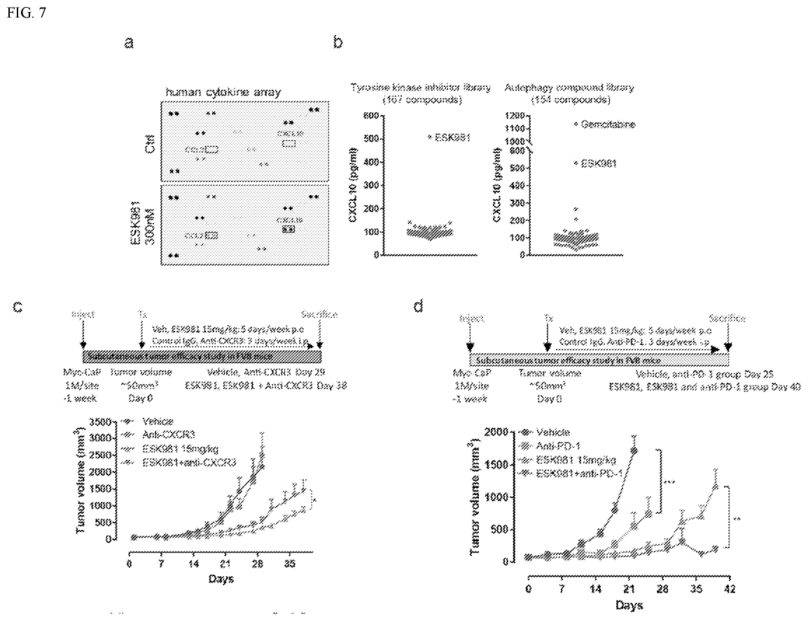

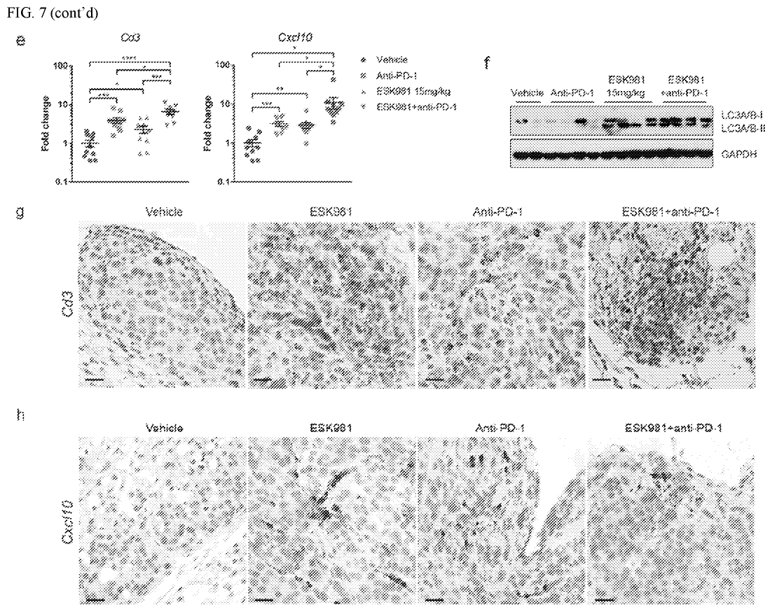

FIG. 7: ESK981 activates an immune response and potentiates the effect of anti-PD-1 immunotherapy in immune-competent murine models.
(a) Human cytokine array using VCaP conditioned medium after 300 nM ESK981 treatment for 24 hours. CXCL10 and CCL2 are highlighted on the dot plots.
(b) CXCL10 protein level was measured by ELISA using conditioned medium from VCaP cells treated with either 300 nM ESK981 or two compound libraries (tyrosine kinase inhibitors and autophagy-related compounds) for 24 hours. N=3 per group. ESK981 is shown in red.
(c) (Top) Schematic illustration of Myc-CaP experimental design in immune-competent mice. p.o, oral gavage. i.p, intraperitoneal. (Bottom) Mice bearing Myc-CaP tumors were randomized into four groups (n=8 per group) for treatment with vehicle, 15 mg/kg ESK981, and/or mouse anti-CXCR3 antibody for 6 weeks. Tumor volumes were measured twice per week with a digital caliper. Data were analyzed by unpaired t test and presented as mean±SEM. *p<0.05.
(d) (Top) Schematic illustration of Myc-CaP experimental design in immune-competent mice. p.o, oral gavage. i.p, intraperitoneal. (Bottom) Mice bearing Myc-CaP tumors were randomized into four groups (n=10 per group) for treatment with vehicle, 15 mg/kg ESK981 (five days per week), and/or mouse anti-PD-1 antibody (three days per week) for 6 weeks. Tumor volumes were measured twice per week with a digital caliper. Data were analyzed by unpaired t test and presented as mean±SEM. **p<0.01; ***p<0.001.
(e) Cd3 (left graph) and Cxcl10 (right graph) mRNA levels were quantified by qPCR in individual tumors after four weeks of the indicated treatments in Myc-CaP tumors. Data were analyzed by unpaired t test and presented as mean±SEM. *p<0.05; ***p<0.001; ****p<0.0001.
(f) Protein levels of LC3 from representative individual tumors were measured by western blot after five days of the indicated treatment in Myc-CaP tumors.
(g) Representative Cd3 RNA ISH from the indicated Myc-CaP tumors. Scale bar: 50 μm.
(h) Representative Cxcl10 RNA ISH from the indicated Myc-CaP tumors. Scale bar: 50 μm.

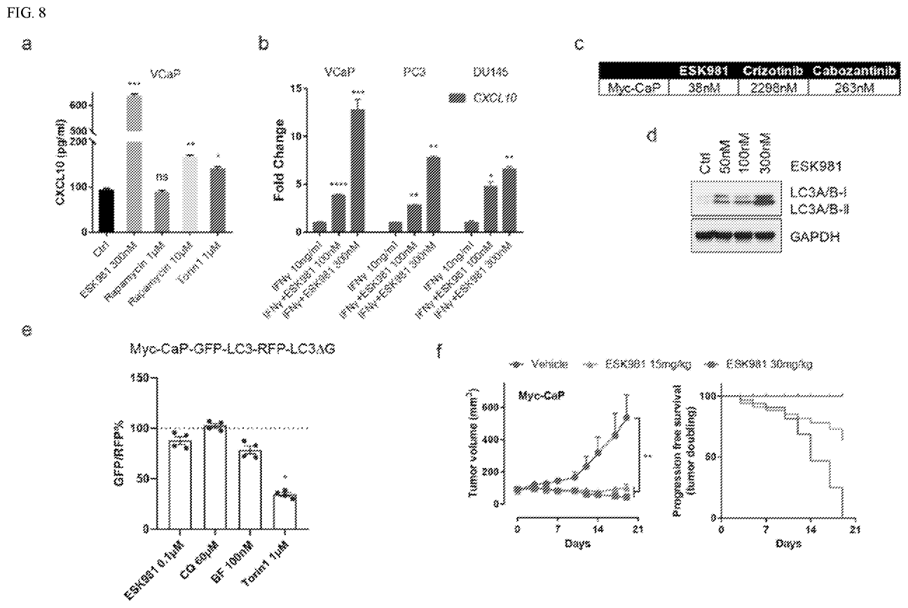

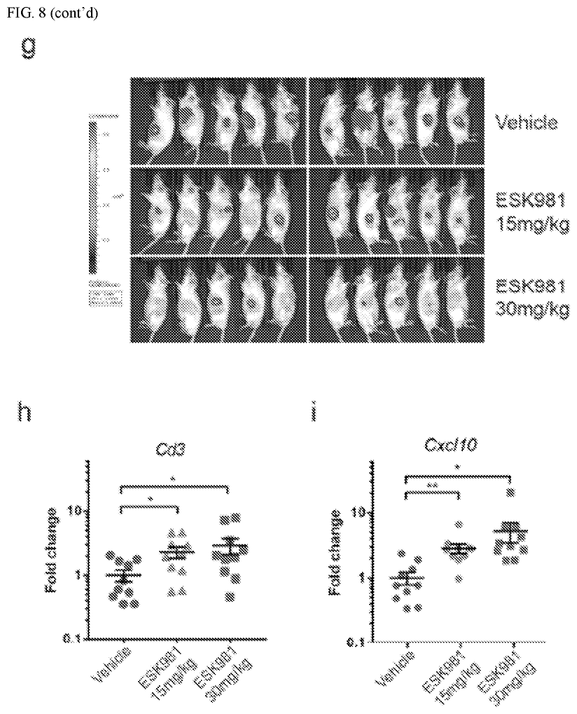

FIG. 8: ESK981 upregulates Cxcl10 expression and inhibits growth of Myc-CaP prostate cancer in immune-competent murine models.
(a) CXCL10 protein levels measured by ELISA in conditioned media from VCaP cells treated with ESK981 or various autophagy inducers for 24 hours. Data were analyzed by unpaired t test and presented as mean±SEM. *p<0.05; **p<0.01; ***p<0.001; ns, p>0.05.
(b) CXCL10 mRNA levels measured by quantitative PCR (qPCR) in VCaP, PC3, and DU145 cells with the indicated treatment for 24 hours. IFNγ, interferon gamma. Data were analyzed by unpaired t test and presented as mean±SEM. *p<0.05; **p<0.01; ***p<0.001; ****p<0.0001.
(c) IC₅₀ of ESK981, crizotinib, and cabozantinib determined in Myc-CaP cells.
(d) Protein levels of LC3 after 50 nM, 100 nM, and 300 nM ESK981 treatment for 24 hours in Myc-CaP cells.
(e) Ratio of GFP/RFP signal in Myc-CaP GFP-LC3-RFP-LC3AG stable expressing cells with the indicated treatment for 24 hours. *p<0.05.
(f) Myc-CaP tumors were established subcutaneously in FVB mice. Mice were randomized into three groups (n=10 per group) once tumors reached an average size of 50 mm³ and were treated with vehicle, 15 mg/kg, or 30 mg/kg ESK981 five days per week for three weeks. Tumor volumes were monitored twice per week. (Right) Progression-free survival (tumor doubling) was calculated from individual tumors. Data were analyzed by unpaired t test and presented as mean±SEM. **p<0.01.
(g) Bioluminescent signaling images were taken from individual Myc-CaP tumor-bearing mice at day 19.
(h) mRNA levels of Cd3 from individual Myc-CaP tumors from the indicated group. Data were analyzed by unpaired t test and presented as mean±SEM. *p<0.05.
(i) Cxcl10 mRNA levels were quantified by qPCR in individual tumors. Data were analyzed by unpaired t test and presented as mean±SEM. *p<0.05; **p<0.01.

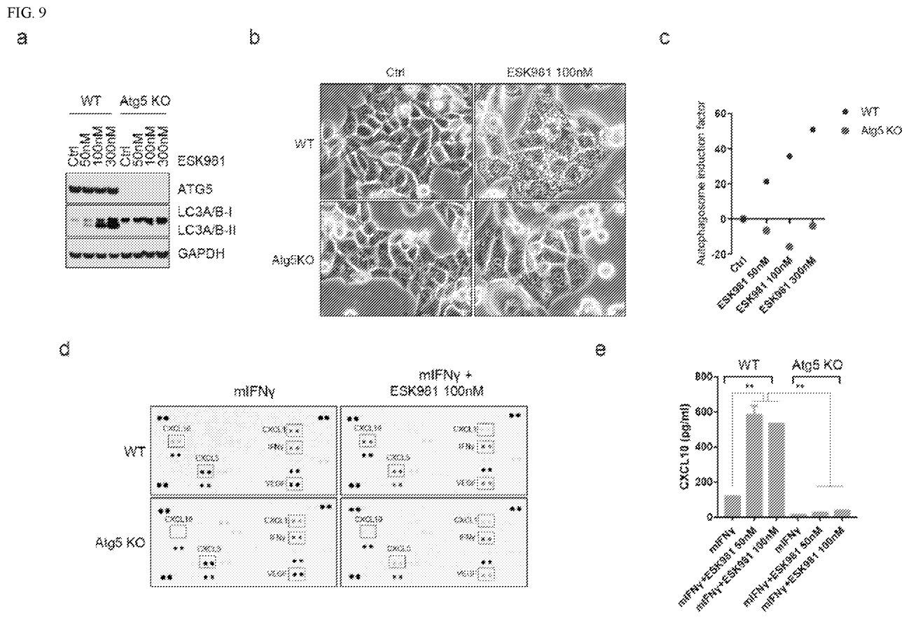

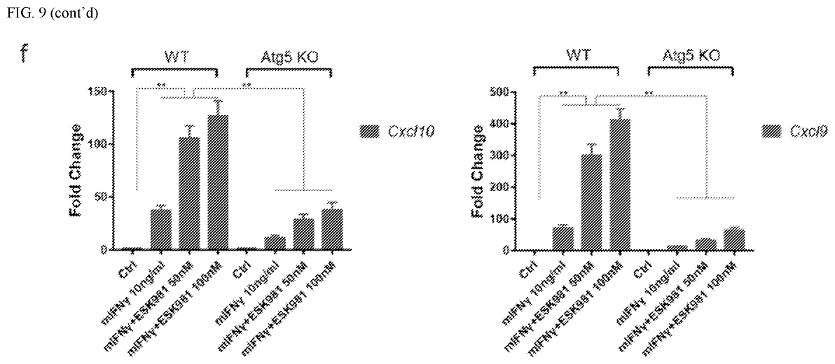

FIG. 9: ATG5 is required for ESK981-induced vacuolization and CXCL10 mediated immune response.
(a) Myc-CaP wild-type (WT) and Atg5 knockout (Atg5 KO) cells were treated with increasing concentrations of ESK981 for 24 hours. Atg5 and LC3 levels were assessed by western blot. GAPDH served as a loading control.
(b) Representative morphology of vacuolization in Myc-CaP wild-type (WT) and Atg5 knockout (Atg5 KO) cells after treatment with control or 100 nM ESK981 for 24 hours.
(c) Autophagosome content of Myc-CaP WT and Atg5 KO cells were measured by CYTO-ID® assay after being treated with increasing concentrations of ESK981 for 24 hours.
(d) Mouse cytokine array using Myc-CaP WT and Atg5 KO cell supernatant after treatment with 10 ng/ml mouse interferon gamma (mIFNγ) or mIFNγ+ 100 nM ESK981 for 24 hours. Differential expression candidate dots are highlighted by boxes.
(e) Mouse CXCL10 protein levels were measured by ELISA in Myc-CaP WT and Atg5 KO conditioned medium with the indicated treatment for 24 hours. Data were analyzed by unpaired t test and presented as mean±SEM. **p<0.01.
(f) mRNA levels of Cxcl10 and Cxcl9 were measured by qPCR in Myc-CaP WT and Atg5 KO cells with 50 nM or 100 nM ESK981 and 10 ng/ml mIFNγ treatment for 24 hours. N=3 samples per group. Data were analyzed by unpaired t test and presented as mean±SEM. **p<0.01.

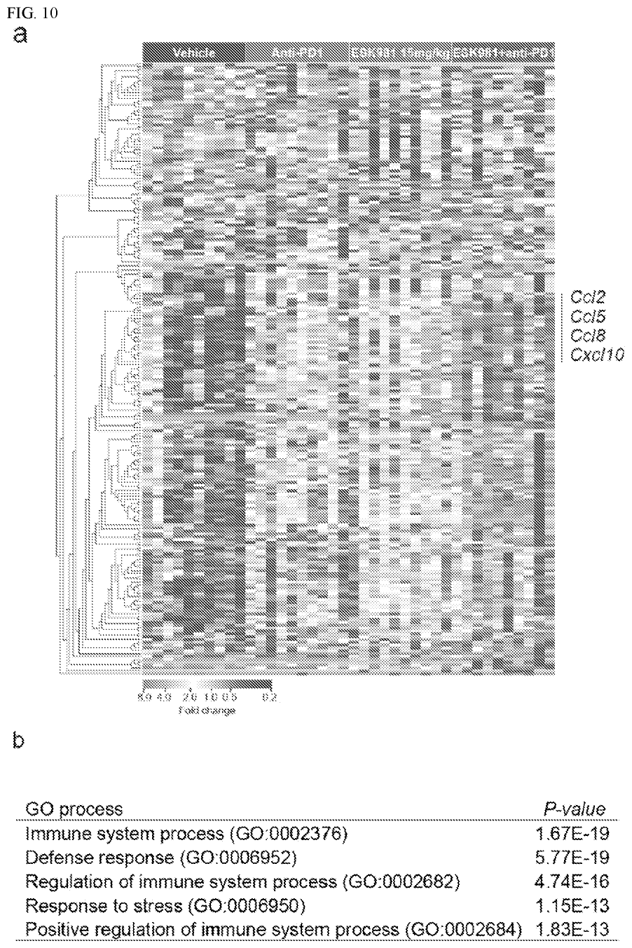

FIG. 10: Transcriptomic analysis of ESK981 in combination with anti-PD-1 immunotherapy in FVB mice.
(a) Heatmap representation of gene expression of individual MyC-CaP tumors treated with either vehicle, anti-PD-1, ESK981, or combination. N=10 tumors per group.
(b) Gene ontology analysis of differentially expressed genes against vehicle group.

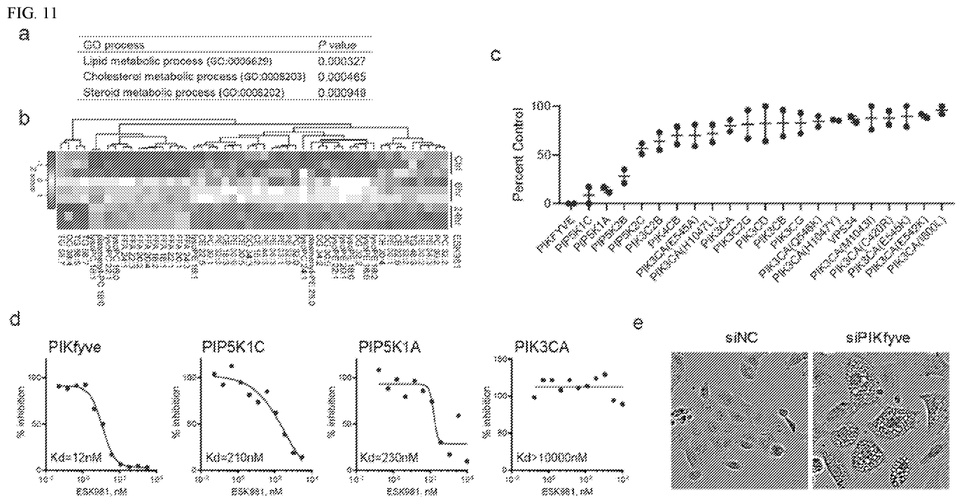

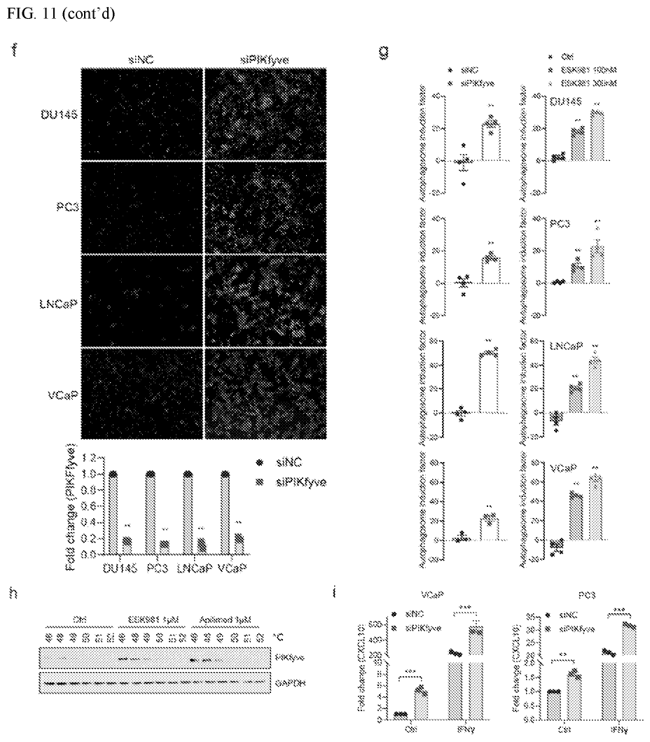

FIG. 11: Identification of lipid kinase PIKfyve as the target of ESK981-induced effects on autophagy and CXCL10 levels.
(a) Gene ontology analysis for top elevated genes after 300 nM ESK981 in VCaP cells for 6 and 24 hour treatments. The top three processes are listed.
(b) Heatmap representation of untargeted lipidomics analysis after 300 nM ESK981 treatment for 6 and 24 hours in VCaP cells. N=5 per group. The PE class is highlighted in red.
(c) Binding affinity of 1 μM ESK981 against a panel of 22 lipid kinases.
(d) Representative dissociation constant (Kd) curve of ESK981 against lipid kinase PIKFYVE, PIP5K1C, PIP5K1A, and PIK3CA.
(e) Morphology of DU145-RFP cells with control siRNA or PIKfyve siRNA.
(f-g) Autophagosome induction activity measured with CYTO-ID® assay in DU145, PC3, LNCaP, and VCaP after siRNA knockdown of PIKfyve or treatment with ESK981 (f, images; g, graphs). Data were analyzed by unpaired t test and presented as mean±SEM. **p<0.01. (0 PIKFYVE mRNA levels were quantified by qPCR in indicated cells after siNC or siPIKfyve knockdown. Data were analyzed by unpaired t test and presented as mean±SEM. **p<0.01.
(h) Cellular thermal shift assay (CESTA) of VCaP cells treated with control, 1 μM ESK981, or 1 μM apilimod for 2 hours.
(i) CXCL10 mRNA levels measured by qPCR in VCaP or PC3 cells after siRNA knockdown of a non-targeting control (siNC) or PIKFYVE (siPIKfyve) with the indicated treatment for 24 hours. Data were analyzed by unpaired t test and presented as mean±SEM. **p<0.01; ***p<0.001.

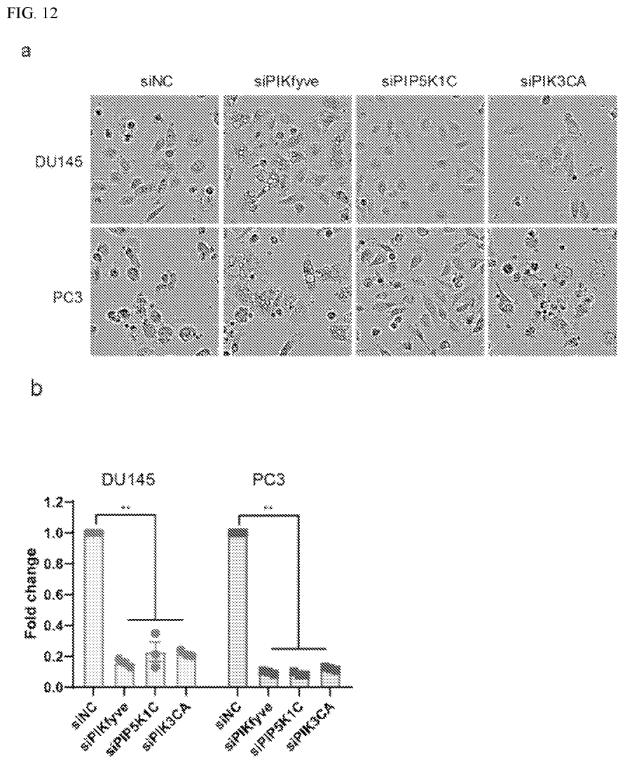

FIG. 12: PIKfyve mediates a cellular vacuolization morphology in prostate cancer cells.
(a) Morphology of DU145 and PC3 cells after siNC, siPIKfyve, siPIP5K1C, or siPIK3CA transfection.
(b) mRNA levels of PIKFYVE, PIP5K1C, and PIK3CA were measured by qPCR after siRNA of indicated targets in DU145 and PC3 cells. Data were analyzed by unpaired t test and presented as mean±SEM. **p<0.01.

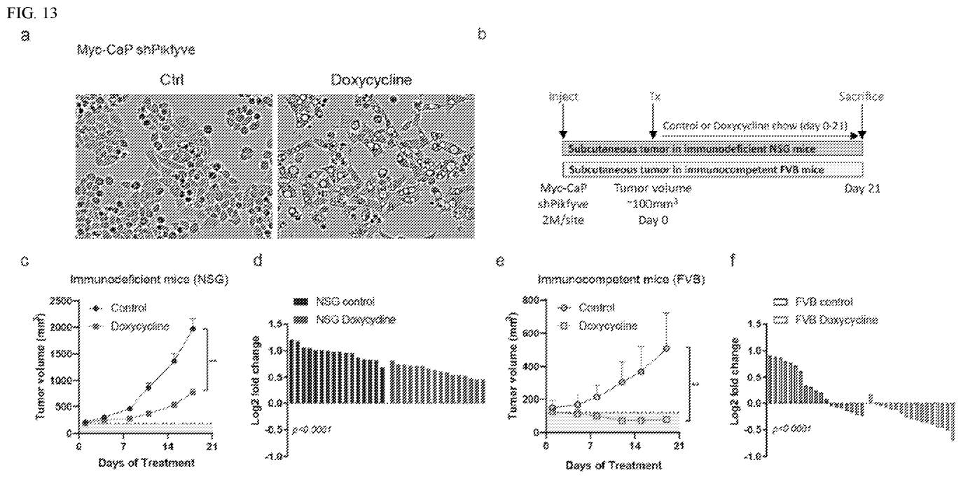

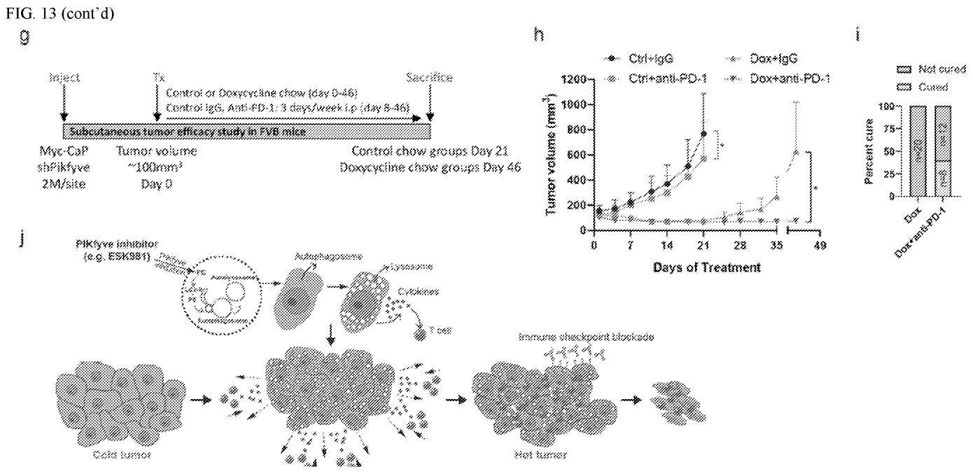

FIG. 13: Genetic inhibition of Pikfyve potentiates the therapeutic benefit of anti-PD-1 immunotherapy in immune-competent murine models.
(a) Representative images of doxycycline inducible shPikfyve Myc-CaP cells with or without 1 μg/ml doxycycline treatment for 72 hours.
(b) Schematic illustration of shPifyve Myc-CaP experimental design in immunodeficient mice (NSG) and immunocompetent mice (FVB).
(c) Average tumor volume of shPikfyve Myc-CaP with or without doxycycline chow in NSG mice. Tumor volumes were measured twice per week with a digital caliper. **p<0.01.
(d) Percent changes in shPikfyve Myc-CaP tumor volume represented by waterfall plot in NSG mice. p<0.0001.
(e) Average tumor volume of shPikfyve Myc-CaP with or without doxycycline chow in FVB mice. Tumor volumes were measured twice per week with a digital caliper. **p<0.01.
(f) Percent changes in shPikfyve Myc-CaP tumor volume represented by waterfall plot in FVB mice. p<0.0001.
(g) Schematic illustration of shPikfyve Myc-CaP experimental design in immunocompetent mice. i.p, intraperitoneal.
(h) Mice bearing shPikfyve Myc-CaP tumors were randomized into four groups (n=10 per group) for treatment with control chow or doxycycline chow, and/or mouse control IgG or anti-PD-1 antibody (three days per week) for 6 weeks. Tumor volumes were measured twice per week with a digital caliper. Data were analyzed by unpaired t test and presented as mean±SEM. *p<0.05.
(i) Percentage cure rate defined as ratio of complete tumor regression on groups of doxycycline chow and/or mouse anti-PD-1 antibody (n=20 per group).
(j) Model of ESK981's mechanism of action and its anti-tumor activity, described in the main text.

### Definitions

The term “anticancer agent” as used herein, refer to any therapeutic agent (e.g., chemotherapeutic compounds and/or molecular therapeutic compounds), antisense therapies, radiation therapies, or surgical interventions, used in the treatment of hyperproliferative diseases such as cancer (e.g., in mammals, e.g., in humans).

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent, an animal or a cell within an animal more susceptible, or more responsive, to the biological effects (e.g., promotion or retardation of an aspect of cellular function including, but not limited to, cell division, cell growth, proliferation, invasion, angiogenesis, necrosis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least 300%, at least about 350%, at least about 400%, at least about 450%, or at least about 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, non-limiting examples of which include, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “neoplastic disease,” as used herein, refers to any abnormal growth of cells being either benign (non-cancerous) or malignant (cancerous).

The term “normal cell,” as used herein, refers to a cell that is not undergoing abnormal growth or division. Normal cells are non-cancerous and are not part of any hyperproliferative disease or disorder.

The term “anti-neoplastic agent,” as used herein, refers to any compound that retards the proliferation, growth, or spread of a targeted (e.g., malignant) neoplasm.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

### DETAILED DESCRIPTION OF THE INVENTION

Multi-tyrosine kinase inhibitors (MTKIs) have had a major impact in the treatment of advanced cancer patients. Experiments conducted during the course of developing embodiments for the present invention screened 167 TKIs for compounds with potential utility in the treatment of castration-resistant prostate cancer (CRPC) and identified ESK981 as a phase I-cleared MTKI with superior efficacy in diverse preclinical models of CRPC, which furthermore exhibited an unexpected accumulation of autophagosome and lysosome levels resulting from inhibition of autophagic flux. When compared against a panel of 154 autophagy-associated compounds and 167 TKIs, ESK981 emerged as the most potent inducer of autophagosome levels. Since autophagy has been linked to secretory processes and the release of cytokines into the tumor microenvironment, experiments were conducted that analyzed levels of cytokines in the presence of ESK981 and found that it increased expression of the Th1-type chemokine CXCL10 in an ATG5-dependent manner. Increased expression of CXCL10 was associated with increased T cell tumor infiltration in syngeneic prostate tumor-bearing mice and enhanced activity of immune checkpoint blockade with ESK981 co-treatment. Furthermore, ESK981 significantly upregulated production of lipids, including phosphatidylethanolamine, and directly targeted the lipid kinase PIKfyve to impact the autophagy pathway. Similar to ESK981, inducible knockdown of PIKfyve in vivo enhanced the activity of immune checkpoint blockade. Taken together, these data indicate that compounds that target autophagy via PIKfyve inhibition potentiates the effects of immune checkpoint blockade in the treatment of advanced prostate cancers.

Accordingly, the present invention provides compositions and methods for preventing, attenuating, or treating disorders characterized with characterized with PIKfyve-expressing cells. In particular, provided herein are methods for preventing, attenuating, or treating disorders characterized with PIKfyve-expressing cells through use of compositions comprising a therapeutic agent capable of inhibiting PIKfyve activity.

In certain embodiments, the present invention provides methods for inhibiting PIKfyve activity in a subject having PIKfyve-expressing cells through administering to the subject a composition comprising a therapeutically effective amount of an agent capable of inhibiting PIKfyve activity (e.g., ESK981 or a compound similar to ESK981).

In certain embodiments, the present invention provides methods for inhibiting conversion of phosphatidylinositol 3-phosphate (PI(3)P) to phosphatidylinositol 3,5-bisphosphate (PI(3,5)P₂) in a subject having PIKfyve-expressing cells through administering to the subject a composition comprising a therapeutically effective amount of an agent capable of inhibiting PIKfyve activity (e.g., ESK981 or a compound similar to ESK981).

In certain embodiments, the present invention provides methods for inhibiting PIKfyve activity related tumor growth in a subject having PIKfyve-expressing cells (e.g., PIKfyve-expressing cancer cells) through administration to the subject a therapeutically effective amount of an agent capable of inhibiting PIKfyve activity (e.g., ESK981 or a compound similar to ESK981).

In certain embodiments, the present invention provides methods for inhibiting PIKfyve activity related autophagic flux in a subject having PIKfyve-expressing cells through administration to the subject a therapeutically effective amount of an agent capable of inhibiting PIKfyve activity (e.g., ESK981 or a compound similar to ESK981).

In certain embodiments, the present invention provides methods for activating an anti-tumor immune response in a subject having PIKfyve-expressing cells through administration to the subject a therapeutically effective amount of an agent capable of inhibiting PIKfyve activity (e.g., ESK981 or a compound similar to ESK981).

In certain embodiments, the present invention provides methods for inhibiting conversion of phosphatidylinositol 3-phosphate (PI(3)P) to phosphatidylinositol 3,5-bisphosphate (PI(3,5)P₂) in PIKfyve-expressing cells through exposing such cells to compositions comprising an agent capable of inhibiting PIKfyve activity (e.g., ESK981 or a compound similar to ESK981).

In certain embodiments, the present invention provides methods for inhibiting PIKfyve activity in PIKfyve-expressing cells through exposing such cells to compositions comprising an agent capable of inhibiting PIKfyve activity (e.g., ESK981 or a compound similar to ESK981).

In certain embodiments, the present invention provides methods for inhibiting PIKfyve activity related tumor growth in PIKfyve-expressing cells (e.g., PIKfyve-expressing cancer cells) through exposing such cells to compositions comprising an agent capable of inhibiting PIKfyve activity (e.g., ESK981 or a compound similar to ESK981).

In certain embodiments, the present invention provides methods for inhibiting PIKfyve activity related autophagic flux in PIKfyve-expressing cells through exposing such cells to compositions comprising an agent capable of inhibiting PIKfyve activity (e.g., ESK981 or a compound similar to ESK981).

In certain embodiments, the present invention provides methods for activating an anti-tumor immune response in cells having increased PIKfyve activity through exposing such cells to compositions comprising an agent capable of inhibiting PIKfyve activity (e.g., ESK981 or a compound similar to ESK981).

The embodiments of the present invention are not limited to specific certain agents capable of inhibiting PIKfyve activity. In some embodiments, the agent is any type or kind of moiety (e.g., small molecule, polypeptide or peptide fragment, antibody or fragment thereof, nucleic acid molecule (e.g., RNA, siRNA, microRNA, interference RNA, mRNA, replicon mRNA, RNA-analogues, and DNA), etc.) capable of inhibiting PIKfyve activity. In some embodiments, the agent is any type or kind of moiety (e.g., small molecule, polypeptide or peptide fragment, antibody or fragment thereof, nucleic acid molecule (e.g., RNA, siRNA, microRNA, interference RNA, mRNA, replicon mRNA, RNA-analogues, and DNA), etc.) capable of inhibiting conversion of phosphatidylinositol 3-phosphate (PI(3)P) to phosphatidylinositol 3,5-bisphosphate (PI(3,5)P₂), inhibiting PIKfyve activity related tumor growth, inhibiting PIKfyve activity related autophagic flux, and/or activating an anti-tumor immune response in cells having increased PIKfyve activity.

In some embodiments, the agent is ESK981 or a compound similar to ESK981, or a pharmaceutically acceptable salt, solvate, or prodrug thereof. In some embodiments, the agent is capable of inhibiting conversion of phosphatidylinositol 3-phosphate (PI(3)P) to phosphatidylinositol 3,5-bisphosphate (PI(3,5)P₂). In some embodiments, the agent is capable of inhibiting PIKfyve activity related tumor growth. In some embodiments, the agent is capable of inhibiting PIKfyve activity related autophagic flux. In some embodiments, the agent is capable of activating an anti-tumor immune response in cells having increased PIKfyve activity.

In certain embodiments, the present invention provides a method of treating cancer in a patient in need thereof, the method comprising administering a therapeutically effective amount of ESK981, or a pharmaceutically acceptable composition thereof, and a therapeutically effective amount of an immune checkpoint inhibitor, or a pharmaceutically acceptable composition thereof, to the patient.

In some embodiments, the immune checkpoint inhibitor is a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

In some embodiments, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, pidilzumab, and cemiplimab-rwlc. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See, e.g., Unanue, E. R., PNAS 110:10886-87 (2013).

In another embodiment, the immune checkpoint inhibitor is a TIGIT inhibitor. See, e.g., Harjunpää 1 and Guillerey, Clin Exp Immunol 200:108-119 (2019).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

In another embodiment, the immune checkpoint inhibitor is a polypeptide that binds to and blocks PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

In another embodiment, the immune checkpoint inhibitor is a compound with peptide moieties that inhibit PD-1 signaling. Examples of such compounds are disclosed in U.S. Pat. No. 8,907,053.

In another embodiment, the immune checkpoint inhibitor is an inhibitor of certain metabolic enzymes, such as indoleamine 2,3 dioxygenase (IDO), which is expressed by infiltrating myeloid cells and tumor cells, and isocitrate dehydrogenase (IDH), which is mutated in leukemia cells. Mutants of the IDH enzyme lead to increased levels of 2-hydroxyglutarate (2-HG), which prevent myeloid differentiation. Stein et al., Blood 130:722-31 (2017); Wouters, Blood 130:693-94 (2017). Particular mutant IDH blocking agents include, but are not limited to, ivosidenib and enasidenib mesylate. Dalle and DiNardo, Ther Adv Hematol 9(7):163-73 (2018); Nassereddine et al., Onco Targets Ther 12:303-08 (2018). The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to, levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In another embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In another embodiment, the immune checkpoint inhibitor is pembrolizumab, nivolumab, cemiplimab, atezolizumab, avelumab, durvalumab, or ipilimumab.

In another embodiment, the immune checkpoint inhibitor is nivolumab.

ESK981 and the immune checkpoint inhibitor can be administered to the patient together as a single-unit dose or separately as multi-unit doses in any order and by any suitable route of administration.

In one embodiment, ESK981 is administered to the patient before the immune checkpoint inhibitor, e.g., 0.5, 1, 2, 3, 4, 5, 10, 12, or 18 hours, 1, 2, 3, 4, 5, or 6 days, or 1, 2, 3, or 4 weeks prior to the administration of the immune checkpoint inhibitor.

In another embodiment, ESK981 is administered to the patient after the immune checkpoint inhibitor, e.g., 0.5, 1, 2, 3, 4, 5, 10, 12, or 18 hours, 1, 2, 3, 4, 5, or 6 days, or 1, 2, 3, or 4 weeks after the administration of the immune checkpoint inhibitor.

In another embodiment, ESK981 is administered to the subject at the same time as the immune checkpoint inhibitor.

In another embodiment, ESK981 and the immune checkpoint inhibitor to the subject is synergistically effective to treat cancer in the subject.

In another embodiment, ESK981 is administered to the patient according to an intermittent dosing schedule, e.g., for five consecutive days followed by two days off.

In another embodiment, ESK981 is administered orally to the patient.

In another embodiment, the immune checkpoint inhibitor is administered to the patient according to an intermittent dosing schedule, e.g., once a week, once every two weeks, once every three weeks, or once every four weeks.

In another embodiment, the immune checkpoint inhibitor is subcutaneously or intravenously administered to the patient.

In another embodiment, the cancer is prostate cancer, pancreatic cancer, colon cancer, melanoma, lung cancer, breast cancer, renal cancer, lymphoma, ovarian cancer, bladder cancer, Merkel cell carcinoma, rhabdomyosarcoma, osteosarcoma, synovial sarcoma, glioblastoma, Ewing's sarcoma, diffuse intrinsic pontine glioma (DIPG), neuroblastoma, or Wilms' tumor.

In another embodiment, the cancer is metastatic castration resistant prostate cancer.

The compounds of the invention are useful for the treatment, amelioration, or prevention of disorders, such as any type of cancer characterized with PIKfyve-expressing cells and additionally any cells responsive to induction of apoptotic cell death (e.g., disorders characterized by dysregulation of apoptosis, including hyperproliferative diseases such as cancer).

The invention also provides the use of such PIKfyve inhibiting agents to induce cell cycle arrest and/or apoptosis in cells having increased PIKfyve activity (e.g., cancer cells having increased PIKfyve activity). The invention also relates to the use of compounds for sensitizing cells to additional agent(s), such as inducers of apoptosis and/or cell cycle arrest, and chemoprotection of normal cells through the induction of cell cycle arrest prior to treatment with chemotherapeutic agents.

The PIKfyve inhibiting agents are useful for the treatment, amelioration, or prevention of disorders, such as any type of cancer characterized with increased PIKfyve activity (e.g., prostate cancer characterized with PIKfyve-expressing cells).

In certain embodiments, the PIKfyve inhibiting agents can be used to treat, ameliorate, or prevent a cancer characterized with PIKfyve-expressing cells that additionally is characterized by resistance to cancer therapies (e.g., those cancer cells which are chemoresistant, radiation resistant, hormone resistant, and the like). In certain embodiments, the cancer is one or more of prostate cancer, castration resistant prostate cancer, pancreatic cancer, colon cancer, melanoma, lung cancer, breast cancer, renal cancer, lymphoma, ovarian cancer, bladder cancer, Merkel cell carcinoma, rhabdomyosarcoma, osteosarcoma, synovial sarcoma, glioblastoma, Ewing's sarcoma, diffuse intrinsic pontine glioma (DIPG), neuroblastoma, and Wilms' tumor.

In such embodiments, the compounds inhibit the activity of PIKfyve which results in inhibited growth of PIKfyve-expressing cancer cells or supporting cells outright and/or render such cells as a population more susceptible to the cell death-inducing activity of cancer therapeutic drugs (e.g., immune checkpoint inhibitors) or radiation therapies. In some embodiments, the inhibition of PIKfyve-expressing cancer cells activity occurs through, for example, inhibiting conversion of phosphatidylinositol 3-phosphate (PI(3)P) to phosphatidylinositol 3,5-bisphosphate (PI(3,5)P₂), inhibiting PIKfyve activity related tumor growth, inhibiting PIKfyve activity related autophagic flux, and/or activating an anti-tumor immune response in cells having increased PIKfyve activity.

In some embodiments, one or more anticancer agents are co-administered with the PIKfyve inhibiting agent, wherein said anticancer agent one or more of an immune checkpoint inhibitor (e.g., pembrolizumab, nivolumab, cemiplimab, atezolizumab, avelumab, durvalumab, ipilimumab), a chemotherapeutic agent, and radiation therapy.

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-κB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); antiandrogens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a described agent capable of inhibiting PIKfyve activity (e.g., ESK981 or compounds structurally similar to ESK981) and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., Emeryville, CA |
| (des-alanyl-1, serine-125 human interleukin-2) | | |
| Alemtuzumab | Campath | Millennium and ILEX |
| (IgG1κ anti CD52 antibody) | | Partners, LP, Cambridge, MA |
| Alitretinoin | Panretin | Ligand Pharmaceuticals, Inc., |
| (9-cis-retinoic acid) | | San Diego CA |
| Allopurinol | Zyloprim | GlaxoSmithKline, Research |
| (1,5-dihydro-4H-pyrazolo[3,4-d]pyrimidin-4-one | | Triangle Park, NC |
| monosodium salt) | | |
| Altretamine | Hexalen | US Bioscience, West |
| (N,N,N′,N′,N″,N″,-hexamethyl-1,3,5-triazine-2,4,6- | | Conshohocken, PA |
| triamine) | | |
| Amifostine | Ethyol | US Bioscience |
| (ethanethiol, 2-[(3-aminopropyl)amino]-, dihydrogen | | |
| phosphate (ester)) | | |
| Anastrozole | Arimidex | AstraZeneca Pharmaceuticals, |
| (1,3-Benzenediacetonitrile, a,a,a′,a′-tetramethyl-5-(1H- | | LP, Wilmington, DE |
| 1,2,4-triazol-1-ylmethyl)) | | |
| Arsenic trioxide | Trisenox | Cell Therapeutic, Inc., Seattle, |
| | | WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain of | | Durham, NC |
| Mycobacterium bovis (Bacillus Calmette-Gukin [BCG], | | |
| substrain Montreal) | | |
| bexarotene capsules | Targretin | Ligand Pharmaceuticals |
| (4-[1-(5,6,7,8-tetrahydro-3,5,5,8,8-pentamethyl-2- | | |
| napthalenyl) ethenyl] benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics produced by | | NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]-cytidine) | | |
| Carboplatin | Paraplatin | Bristol-Myers Squibb |
| (platinum, diammine [1,1-cyclobutanedicarboxylato(2-)- | | |
| 0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford Pharmaceuticals, |
| | | Inc., Baltimore, MD |
| Celecoxib | Celebrex | Searle Pharmaceuticals, |
| (as 4-[5-(4-methylphenyl)-3-(trifluoromethyl)-1H- | | England |
| pyrazol-1-yl] benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson Pharmaceutical |
| (2-chloro-2′-deoxy-b-D-adenosine) | | Research Institute, Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC-Dome | Bayer AG, Leverkusen, |
| (5-(3,3-dimethyl-1-triazeno)-imidazole-4-carboxamide | | Germany |
| (DTIC)) | | |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., Thousand Oaks, |
| (recombinant peptide) | | CA |
| daunorubicin liposomal | DanuoXome | Nexstar Pharmaceuticals, Inc., |
| ((8S-cis)-8-acetyl-10-[(3-amino-2,3,6-trideoxy-á-L-lyxo- | | Boulder, CO |
| hexopyranosyl)oxy]-7,8,9,10-tetrahydro-6,8,11- | | |
| trihydroxy-1-methoxy-5,12-naphthacenedione | | |
| hydrochloride) | | |
| Daunorubicin HCl, daunomycin | Cerubidine | Wyeth Ayerst, Madison, NJ |
| ((1S,3S)-3-Acetyl-1,2,3,4,6,11-hexahydro-3,5,12- | | |
| trihydroxy-10-methoxy-6,11-dioxo-1-naphthacenyl 3- | | |
| amino-2,3,6-trideoxy-(alpha)-L-lyxo-hexopyranoside | | |
| hydrochloride) | | |
| Denileukin diftitox | Ontak | Seragen, Inc., Hopkinton, MA |
| (recombinant peptide) | | |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, Inc., |
| ((2R,3S)-N-carboxy-3-phenylisoserine, N-tert-butyl ester, | | Bridgewater, NJ |
| 13-ester with 5b-20-epoxy-12a,4,7b,10b,13a- | | |
| hexahydroxytax-11-en-9-one 4-acetate 2-benzoate, | | |
| trihydrate) | | |
| Doxorubicin HCl | Adriamycin, Rubex | Pharmacia & Upjohn |
| (8S,10S)-10-[(3-amino-2,3,6-trideoxy-a-L-lyxo- | | Company |
| hexopyranosyl)oxy]-8-glycolyl-7,8,9,10-tetrahydro- | | |
| 6,8,11-trihydroxy-1-methoxy-5,12-naphthacenedione | | |
| hydrochloride) | | |
| doxorubicin | Adriamycin PFS | Pharmacia & Upjohn |
| | Intravenous injection | Company |
| doxorubicin liposomal | Doxil | Sequus Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one propionate) | | Indianapolis, IN |
| dromostanolone propionate | Masterone injection | Syntex, Corp., Palo Alto, CA |
| Elliott's B Solution | Elliott's B Solution | Orphan Medical, Inc |
| Epirubicin | Ellence | Pharmacia & Upjohn |
| ((8S-cis)-10-[(3-amino-2,3,6-trideoxy-a-L-arabino- | | Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3-[bis(2- | | Company |
| chloroethyl)carbamate] 17-(dihydrogen phosphate), | | |
| disodium salt, monohydrate, or estradiol 3-[bis(2- | | |
| chloroethyl)carbamate] 17-(dihydrogen phosphate), | | |
| disodium salt, monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′-(dihydrogen | | |
| phosphate)) | | |
| etoposide, VP-16 | Vepesid | Bristol-Myers Squibb |
| (4′-demethylepipodophyllotoxin 9-[4,6-0-(R)-ethylidene- | | |
| (beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral agent | | Cedar Knolls, NJ |
| vidarabine, 9-b-D-arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta fluoropentylsulphinyl) | | Guayama, Puerto Rico |
| nonyl]estra-1,3,5-(10)-triene-3,17-beta-diol) | | |
| Gemcitabine | Gemzar | Eli Lilly |
| (2′-deoxy-2′,2′-difluorocytidine monohydrochloride (b- | | |
| isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea covalent | | Cambridge MA |
| bond between the monoclonal antibody Ibritumomab and | | |
| the linker-chelator tiuxetan [N-[2- | | |
| bis(carboxymethyl)amino]-3-(p-isothiocyanatophenyl)- | | |
| propyl]-[N-[2-bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino-2,3,6- | | Company |
| trideoxy-(alpha)-L-lyxo-hexopyranosyl)oxy]-7,8,9,10- | | |
| tetrahydro-6,9,11-trihydroxyhydrochloride, (7S-cis)) | | |
| Ifosfamide | IFEX | Bristol-Myers Squibb |
| (3-(2-chloroethyl)-2-[(2-chloroethyl)amino]tetrahydro- | | |
| 2H-1,3,2-oxazaphosphorine 2-oxide) | | |
| Imatinib Mesilate | Gleevec | Novartis AG, Basel, |
| (4-[(4-Methyl-1-piperazinyl)methyl]-N-[4-methyl-3-[[4- | | Switzerland |
| (3-pyridinyl)-2-pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A (Lyophilized | Schering AG, Berlin, |
| (recombinant peptide) | Betaseron) | Germany |
| Irinotecan HCl | Camptosar | Pharmacia & Upjohn |
| ((4S)-4,11-diethyl-4-hydroxy-9-[(4- | | Company |
| piperidinopiperidino)carbonyloxy]-1H-pyrano[3′,4′:6,7] | | |
| indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) dibenzonitrile) | | |
| Leucovorin | Wellcovorin, Leucovorin | Immunex, Corp., Seattle, WA |
| (L-Glutamic acid, N[4[[(2amino-5-formyl-1,4,5,6,7,8 | | |
| hexahydro4oxo6-pteridinyl)methyl]amino]benzoyl], | | |
| calcium salt (1:1)) | | |
| Levamisole HCl | Ergamisol | Janssen Research Foundation, |
| ((−)-(S)-2,3,5,6-tetrahydro-6-phenylimidazo [2,1-b] | | Titusville, NJ |
| thiazole monohydrochloride C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene-3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L-glutamic | | |
| acid) | | |
| Methoxsalen | Uvadex | Therakos, Inc., Way Exton, Pa |
| (9-methoxy-7H-furo[3,2-g][1]-benzopyran-7-one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p-chlorophenyl) | | |
| ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10-anthracenedione | | |
| dihydrochloride) | | |
| Nandrolone phenpropionate | Durabolin-50 | Organon, Inc., West Orange, |
| | | NJ |
| Nofetumomab | Verluma | Boehringer Ingelheim Pharma |
| | | KG, Germany |
| Oprelvekin | Neumega | Genetics Institute, Inc., |
| (IL-11) | | Alexandria, VA |
| Oxaliplatin | Eloxatin | Sanofi Synthelabo, Inc., NY, NY |
| (cis-[(1R,2R)-1,2-cyclohexanediamine-N,N′] | | |
| [oxalato(2-)-O,O′] platinum) | | |
| Paclitaxel | TAXOL | Bristol-Myers Squibb |
| (5β,20-Epoxy-1,2a,4,7β,10β,13a-hexahydroxytax-11- | | |
| en-9-one 4,10-diacetate 2-benzoate 13-ester with (2R,3S)- | | |
| N-benzoyl-3-phenylisoserine) | | |
| Pamidronate | Aredia | Novartis |
| (phosphonic acid (3-amino-1-hydroxypropylidene) bis-, | | |
| disodium salt, pentahydrate, (APD)) | | |
| Pegademase | Adagen (Pegademase | Enzon Pharmaceuticals, Inc., |
| ((monomethoxypolyethylene glycol succinimidyl) 11-17- | Bovine) | Bridgewater, NJ |
| adenosine deaminase) | | |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl human G- | | |
| CSF (Filgrastim) and monomethoxypolyethylene glycol) | | |
| Pentostatin | Nipent | Parke-Davis Pharmaceutical |
| | | Co., Rockville, MD |
| Pipobroman | Vercyte | Abbott Laboratories, Abbott |
| | | Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, Inc., |
| | | Vancouver, Canada |
| Procarbazine | Matulane | Sigma Tau Pharmaceuticals, |
| (N-isopropyl-μ-(2-methylhydrazino)-p-toluamide | | Inc., Gaithersburg, MD |
| monohydrochloride) | | |
| Quinacrine | Atabrine | Abbott Labs |
| (6-chloro-9-(1-methyl-4-diethyl-amine) butylamino-2- | | |
| methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)-D- | | |
| glucopyranose and 220 mg citric acid anhydrous) | | |
| Talc | Sclerosol | Bryan, Corp., Woburn, MA |
| (Mg₃Si₄O₁₀ (OH)₂) | | |
| Tamoxifen | Nolvadex | AstraZeneca Pharmaceuticals |
| ((Z)2-[4-(1,2-diphenyl-1-butenyl) phenoxy]-N,N- | | |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as-tetrazine- | | |
| 8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien-17-oic | | |
| acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine,1,1′,1″-phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl-4,9-dihydroxy- | | |
| 1H-pyrano[3′,4′:6,7] indolizino [1,2-b] quinoline-3,14- | | |
| 4H,12H)-dione monohydrochloride) | | |
| Toremifene | Fareston | Roberts Pharmaceutical Corp., |
| (2-(p-[(Z)-4-chloro-1,2-diphenyl-1-butenyl]-phenoxy)- | | Eatontown, NJ |
| N,N-dimethylethylamine citrate (1:1)) | | |
| Tositumomab, I 131 Tositumomab | Bexxar | Corixa Corp., Seattle, WA |
| (recombinant murine immunotherapeutic monoclonal | | |
| IgG₂ₐ lambda anti-CD20 antibody (I 131 is a | | |
| radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard Capsules | Roberts Labs |
| Valrubicin, N-trifluoroacetyladriamycin-14-valerate | Valstar | Anthra --> Medeva |
| ((2S-cis)-2-[1,2,3,4,6,11-hexahydro-2,5,12-trihydroxy-7 | | |
| methoxy-6,11-dioxo-[[4 2,3,6-trideoxy-3- | | |
| [(trifluoroacetyl)-amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′-norvincaleukoblastine [R- | | |
| (R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS -9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present invention provides methods for administering the described agents capable of inhibiting PIKfyve activity (e.g., ESK981 or compounds structurally similar to ESK981) with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to an animal is about 0.01 Gray (Gy) to about 100 Gy. In another embodiment, about 10 Gy to about 65 Gy (e.g., about Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a described agent capable of inhibiting PIKfyve activity (e.g., ESK981 or compounds structurally similar to ESK981) and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the described agents capable of inhibiting PIKfyve activity (e.g., ESK981 or compounds structurally similar to ESK981) are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the described agents capable of inhibiting PIKfyve activity (e.g., ESK981 or compounds structurally similar to ESK981) may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example demonstrates that ESK981 inhibits prostate cancer growth in vitro and in vivo and induces a unique vacuolization morphology.

To determine whether MTKIs other than cabozantinib may have the potential to be repositioned for CRPC treatment, a cell viability screen employing a 167-compound library of tyrosine kinase inhibitors was performed in DU145 prostate cancer cells (FIG. 1a). From this screen, ESK981 was identified as a top candidate MTKI that decreased cell viability. ESK981 exhibited potent growth inhibition at the concentration of 300 nM, an effect comparable to SRC inhibitors (KX2-391, dasatinib)²⁴ and the HER2 (ERBB2) inhibitor mubritinib²⁵, compounds that have been previously reported to target DU145 cells. Conversely, crizotinib and cabozantinib, MTKIs that have both been evaluated clinically in CRPC^{7,8,26}, exhibited no such growth inhibitory effects at comparable concentrations (FIG. 1a). Of note, among the 167 compounds tested, ESK981 uniquely triggered a cytoplasmic vacuolization morphology (FIG. 1b) that prompted further investigation of its efficacy, functional impact, and mechanism of action in prostate cancer.

The sensitivity of diverse prostate cancer cell lines to ESK981 in long-term survival assays was analyzed. ESK981 exhibited growth inhibitory IC₅₀ values ranging from 35 to 192 nM across cell lines including AR positive cells (VCaP, LNCaP, 22RV1, LNCaP-AR) and AR-negative cells (PC3, DU145). In contrast, cabozantinib and crizotinib exhibited micromolar IC₅₀ values (FIG. 1c, FIG. 2a-b). In two enzalutamide-resistant cell lines, LNCaP-AR and CWR-R1, ESK981 remained efficacious (FIG. 1d), indicating that ESK981 may have utility following enzalutamide progression in CRPC. Furthermore, ESK981 sensitivity was tested using an in vitro 3D spheroid culture and quantification system. In this setting, the formed ‘organoid’ mimics the in vivo environment^{27,28}. Using this technique, ESK981 produced a more robust inhibitory effect at relatively lower concentrations than cabozantinib in VCaP 3D spheroid culture (FIG. 1e).

Whether ESK981 impacts other cellular functions in prostate cancer cells in vitro was next assessed. Data from cell cycle analyses indicated that ESK981 induced a dose-dependent G₂/M phase arrest (FIG. 2c-d). In comparison with other inhibitors in VCaP cells, such as cabozantinib (5 μM), crizotinib (3 μM), or enzalutamide (10 μM), ESK981 (100 nM) demonstrated greater G₂/M arrest potential at a lower concentration (FIG. 2d). Additionally, ESK981 inhibited invasion in a dose-dependent manner in four invasive prostate cancer cell lines in vitro (FIG. 2e). These results demonstrated that ESK981 has superior efficacy compared to other MTKIs in prostate cancer in vitro.

To determine whether ESK981-mediated growth inhibition translated into anti-tumor effects, the efficacy of ESK981 in blocking prostate cancer growth in mouse xenograft models was assessed. VCaP cells were chosen for initial experiments because this cell line harbors the TMPRSS2:ERG gene fusion and AR amplification, both of which are frequent molecular aberrations in patients with advanced CRPC²⁹. A castration-resistant VCaP tumor-bearing xenograft mouse model was generated to mimic disease progression in human patients (FIG. 3a). Treatment with ESK981 (30 or 60 mg/kg) resulted in significant dose-dependent growth inhibiton of VCaP xenografts compared to vehicle (FIG. 10. ESK981 treatment also resulted in dose-dependent reductions in castration-resistant VCaP tumor weights (FIG. 3b) and cell proliferation, assessed by Ki67 (MKI67) immunohistochemistry (IHC) (FIG. 3c). To evaluate the toxicity of ESK981 in mice, body weight changes were monitored (FIG. 4a-c), liver and kidney functions were evaluated by serum chemistry (FIG. 4d), and the histology of major organs was assessed (FIG. 4e). ESK981 was well-tolerated in mice, and body weight loss was within the range of tolerance.

Experiments next assessed the efficacy of ESK981 as a monotherapy in three additional prostate preclinical xenograft models at the 30 mg/kg dose in SCID mice: MDA-PCa-146-12, an AR-positive patient-derived xenograft (PDX); DU145, an AR-negative prostate cancer cell line; and MDA-PCa-146-10, an AR-negative and neuroendocrine prostate cancer (NEPC) PDX model. Significant inhibition of tumor growth by ESK981 was observed in all three models (FIG. 1g-i), and this was mirrored by significant decreases in tumor weights (FIG. 3d, f, h) and Ki67 IHC staining (FIG. 3e, g). Furthermore, hematoxylin and eosin (H&E) staining showed that the vacuolization phenomenon was recapitulated in vivo in tumors treated with ESK981 (FIG. 1j, FIG. 4f), and no major organs were found to have the same tissue vacuolization morphology that was observed in tumors (FIG. 4e). These results demonstrate that ESK981 possesses broad anti-tumor potential for major subtypes of advanced prostate cancer while also triggering a tumor vacuolization morphology.

### Example II

This experiment demonstrates that ESK981 induces autophagosome and lysosome accumulation through inhibition of autophagic flux in prostate cancer cells.

Given the unique vacuolization morphology triggered by ESK981 in vitro and in vivo, experiments were conducted that determined whether this compound affected the autophagy pathway as part of its mechanism of action. Autophagy is an evolutionarily conserved, orderly process of degradation and destruction of cellular components. As part of this process, double-membraned vesicles known as autophagosomes are formed by the engulfment of cytoplasmic constituents; the autophagosomes then fuse with lysosomes to form autolysosomes and initiate degradation and recycling of cargo³⁰. The nature of the ESK981-associated cellular vacuolization was investigated in combination with various autophagic pathway inhibitors. The early autophagosome inhibitor 3-methyladenine (3-MA) partially negated the cellular vacuolization effects of ESK981 in DU145 cells (FIG. 5a). The anti-malarial drug chloroquine (CQ) and the vacuolar-type H⁺-ATPase inhibitor bafilomycin A₁ (BF), inhibitors of autophagy and lysosomal fusion³¹, completely blocked the cellular vacuolization effects of ESK981 (FIG. 5a), suggesting that these vacuoles are indeed linked to autophagic processes.

To measure the ability of ESK981 to impact cellular autophagosome content, experiments were conducted that employed the CYTO-ID® assay, an autophagy detection kit that selectively measures autophagosomes with minimal staining of lysosomes³². The quantified fluorescence intensity showed dose-dependent induction of autophagosome signals by ESK981 in LNCaP, VCaP (FIG. 5b), and DU145 cells (FIG. 6a). Levels of autophagosome induction by ESK981 were higher than a known autophagy activator, the mTOR inhibitor rapamycin (FIG. 5b). Using this high-throughput autophagosome detection method, ESK981 was compared with two compound libraries. Among the 154 autophagy-related compounds and the 167 compounds in the tyrosine kinase inhibitor library, ESK981 demonstrated the highest potency at inducing autophagosome levels in DU145 cells (FIG. 5c-d). Most of the top candidates screened from these two libraries were mTOR inhibitors and other well-known kinase inhibitors reported to possess autophagosome induction capability.

As a further measure of autophagosome levels, the state of MAP1LC3A/B (microtubule associated protein 1 light chain 3 alpha/beta; LC3) was assessed³¹. During autophagy, the cytosolic form of LC3 (LC3-I) gets recruited to the phagophore (the autophagosome precursor) membrane where it is conjugated to phosphatidylethanolamine (PE) to generate the lipidated form of LC3, LC3-II. Numerous prostate cancer cell lines were tested and exhibited an increase in the total level of LC3 lipidation in an ESK981 dose-dependent manner (FIG. 5e). Evidence for an increase in LC3-II was seen within three hours of treatment with 300 nM ESK981 in VCaP cells (FIG. 6b). In contrast, cabozantinib did not induce LC3-II, even after 1 μM treatment for 24 hours. Likewise, crizotinib did not induce an increase in LC3-II at 300 nM and only weakly induced an increase in LC3-II at 1 μM (FIG. 6c).

Much of the fundamental understanding of the autophagic process has come from seminal work carried out in yeast³³. To determine whether ESK981 impinged upon a conserved autophagic process, experiments were conducted that utilized a drug permeable yeast strain (prd5Δ) and analyzed Atg8, the yeast homolog to LC3. ESK981 treatment caused an increase in lipidated Atg8 (FIG. 6d) similar to the results seen in prostate cancer cell lines with LC3, suggesting ESK981 has a common target in yeast. However, eukaryotic-like tyrosine kinases are absent in yeast³⁴, which suggested that ESK981-induced autophagosome levels were independent of tyrosine kinase inhibition and likely involved targeting of a different class of kinases.

As another method to analyze autophagosome levels in prostate cancer cells, experiments were conducted that monitored GFP-LC3 puncta formation with ESK981 treatment after various time points. ESK981 induced LC3 puncta formation in DU145 cells within 1 hour of ESK981 treatment (FIG. 5f), prior to ESK981-induced vacuole formation, which was only observed after four hours of treatment. To better visualize subcellular components, experiments were conducted that performed transmission electron microscopy (TEM) on DU145 cells and were able to demonstrate mostly clear vacuoles adjacent to double-membraned autophagic vesicles after 24 hours of ESK981 treatment (FIG. 5g). In concordance with in vitro results, vacuoles containing cellular materials in vivo were observed by employing TEM with ESK981-treated tumor samples (FIG. 5h). Collectively, these results suggested that the large empty vacuoles induced by ESK981 were unlikely to be autophagosomes. Indeed, immunofluorescence showed that the ESK981-induced vacuoles were positive for the lysosomal marker LAMP1 (FIG. 5i). Increased ESK981 lysosome quantity was observed in a dose-dependent manner as viewed by LysoTracker Green and, as shown in four prostate cancer cell lines, was readily neutralized by BF (FIG. 5j).

Increased autophagosome and lysosome levels by ESK981 could be due to either activation of autophagy or inhibition of autophagic flux. To measure autophagic flux in prostate cancer cell lines, experiments were conducted utilized a novel GFP-LC3-RFP-LC3ΔG probe developed by Mizushima³⁵. This probe allows for direct assessment of autophagic flux without being combined with lysosomal inhibitors. When expressed in cells, the GFP-LC3-RFP-LC3AG probe is cleaved into a degradable fragment, GFP-LC3, and a stable fragment, RFP-LC3AG. A decrease of the GFP/RFP ratio indicates the occurrence of high autophagic flux. In GFP-LC3-RFP-LC3AG expressing PC3 or DU145 cells, the mTORC inhibitor Torin1 decreased the GFP/RFP ratio, suggesting high autophagic flux (FIG. 5k). Conversely, ESK981, CQ, and BF all showed low autophagic flux (FIG. 5k). These results indicate that increased autophagosome and lysosome signals are likely due to decreased autophagic flux with ESK981 treatment in prostate cancer cells.

Finally, several autophagic pathway regulators were targeted to determine which were required for autophagosome and lysosome accumulation. ULK1, Beclin1, FIP200, ATG5, and ATG7 were targeted with siRNA in LNCaP and VCaP cells, and only ATG5 consistently blocked ESK981 or sunitinib-induced LC3 lipidation (FIG. 6e). The association between ESK981-induced effects and autophagy was further validated by genetic targeting of Atg5 levels₃₀. Vacuolization was significantly induced by ESK981 in wild-type mouse embryonic fibroblast (MEF) cells but was largely attenuated in autophagy-deficient Atg5^{-/-} MEFs (FIG. 5l). As expected, LC3 lipidation induced by ESK981 treatment was abolished in Atg5 knockout MEFs (FIG. 5l). These results demonstrate that ESK981-induced autophagosome and lysosome accumulation, associated with a vacuolization morphology, are ATG5-dependent and involve blockade of autophagic flux.

### Example III

This example demonstrates that ESK981 increases levels of the CXCL10 chemoattractant and potentiates effects of anti-PD-1 immunotherapy.

Autophagy has been mechanistically linked with cellular secretory processes, including release of cytokines into the tumor microenvironment³⁶. To evaluate whether ESK981-altered autophagic processes involved regulation of the tumor secretome, the secretion of chemokines was analyzed using a human cytokine array consisting of 105 cytokines. ESK981 significantly induced only CXCL10 and, to a lesser extent, CCL2 in VCaP cell conditioned media after 24 hours of treatment with ESK981 (FIG. 7a). A human CXCL10 ELISA assay was used to compare tyrosine kinase inhibitor and autophagy-linked compound libraries to ESK981 at 300 nM in VCaP-conditioned medium. ESK981 ranked first in inducing CXCL10 levels in the tyrosine kinase inhibitor library and second in the autophagy-linked compound library (FIG. 7b). Interestingly, gemcitabine was also identified as a robust CXCL10 inducer in VCaP cells (FIG. 7b). Well known autophagy regulators rapamycin and Torin 1 marginally increased CXCL10 secretion, but only at higher concentrations (FIG. 8a). CXCL10 expression is known to be regulated physiologically by interferon gamma (IFNγ)³⁷. In multiple human prostate cancer cell lines (VCaP, PC3, and DU145), ESK981 was also able to enhance expression of CXCL10 in the presence of IFNγ (FIG. 8b).

CXCL10 was previously described to be involved in recruitment of T cells into human melanoma³⁸, thus suggesting that ESK981 may increase intratumoral T cell levels and exert an immune response through upregulation of chemokine secretion in the tumor microenvionment^{39,40}. Therefore, experiments were conducted that utilized a mouse syngeneic prostate cancer model driven by human MYC expression (Myc-CaP)^{41,42} to investigate the relationship between immune response and ESK981 in the setting of prostate cancer. Experiments were conducted that first characterized the cell line response to ESK981 in vitro. In Myc-CaP cells, ESK981 had a growth inhibitory IC₅₀ value of 35 nM and remained the most efficacious compound in comparison to crizotinib and cabozantinib (FIG. 8c). Accumulation of autophagosome levels by ESK981 treatment was recapitulated in Myc-CaP cells (FIG. 8d), and autophagic flux was also inhibited in GFP-LC3-RFP-LC3ΔG-expressing Myc-CaP cells after ESK981, CQ, or BF treatment (FIG. 8e). Atg5 knockout Myc-CaP cells were further generated using CRISPR, and consistent with data in human prostate cancer cell lines, Atg5 knockout in Myc-CaP cells significantly blocked ESK981-induced LC3 lipidation (FIG. 9a). The lysosome vacuolization morphology and autophagosome levels measured by CYTO-ID® were also decreased compared to parental cells (FIG. 9b-c). Similar to human prostate cancer cell lines, Myc-CaP cells were also able to enhance IFNγ regulation of CXCL10 secretion and expression (and CXCL9) with ESK981 (FIG. 9d-f). Interestingly, however, this phenomenon was diminished in Atg5 knockout cells, further suggesting that CXCL10 levels are indeed directly impacted by the autophagy pathway. Combined, these data indicate that the mechanism of action of ESK981 is consistent between human and mouse prostate cancer models.

Having characterized the Myc-CaP response to ESK981 in vitro, experiments were conducted that next generated Myc-CaP subcutaneous tumors in FVB mice and treated with vehicle or ESK981 (15 mg/kg, 30 mg/kg). Tumors treated with ESK981 demonstrated dose-dependent growth inhibition and significantly increased tumor doubling time (FIG. 80. Tumor burden was quantified by bioluminescence intensity of individual tumors, and the results indicated that five out of ten Myc-CaP tumors were bioluminescent-negative in the ESK981 30 mg/kg group (FIG. 8g). In addition, analysis of mRNA levels of the T cell marker Cd3 showed a dose-dependent upregulation in the ESK981-treated Myc-CaP tumors (FIG. 8h). In concordance with in vitro data, ESK981 treatment also significantly increased average Cxcl10 levels in a dose-dependent manner (FIG. 8i). Accordingly, the growth inhibitory effect mediated by ESK981 was partially negated by neutralizing anti-CXCR3 (the receptor for CXCL10 and CXCL9) antibody (FIG. 7c).

Enhanced Cxcl10 and Cd3 expression in the ESK981 treatment group of the Myc-CaP syngeneic model prompted us to examine whether ESK981 was able to modulate the efficacy of checkpoint blockade immunotherapy. Subcutaneous tumors were established and treated with vehicle, 15 mg/kg ESK981, anti-PD-1 (Pdcd1), or a combination of ESK981 and anti-PD-1. Anti-PD-1 demonstrated a tumor inhibitory effect that was strongly enhanced by ESK981 co-treatment (FIG. 7d). Consistently, Cd3 and Cxcl10 were upregulated in both the ESK981 solo treatment and combination groups (FIG. 7e). Furthermore, groups receiving ESK981 treatment showed elevated LC3 lipidation, confirming autophagosome accumulation in tumors (FIG. 70. As demonstrated by Cd3 RNA in situ hybridization (ISH), anti-PD-1 and ESK981 each increased CD3⁺ T cell trafficking in the tumor microenvironment, and this effect was enhanced by the combination of anti-PD-1 and ESK981 (FIG. 7g). A similar effect was observed for Cxcl10 RNA ISH (FIG. 7h). Immune activation was further confirmed by transcriptomic analysis from individual tumors, which showed upregulation of inflammatory responses and Cxcl10 with combination treatment (FIG. 10). Taken together, these results suggest that ESK981 increases CXCL10 secretion by blocking autophagic flux in tumor cells, resulting in T cell recruitment to the tumor microenvironment and enhanced therapeutic benefit of anti-PD-1 therapy in prostate cancer.

### Example IV

This example demonstrates identification of lipid kinase PIKfyve as the target of ESK981-mediated autophagy inhibition.

To define the mechanism of action and signaling pathway alterations underlying the functional effects of ESK981, transcriptomic changes were analyzed by RNA-seq of VCaP cells treated with ESK981 (300 nM) for 6 and 24 hours. The resulting data confirmed that ESK981 is involved in modulating an immune response since the top genes upregulated by ESK981 in a time-dependent manner were the Th1-type chemokines CXCL10 and CCL2. Notably, the remaining genes belonged to lipid, cholesterol, and steroid metabolic processes (FIG. 11a), indicating that ESK981 may also play a role in cellular lipid production. Untargeted lipidomic analysis performed in VCaP cells with the same conditions used for RNA-seq demonstrated that phosphatidylethanolamine (PE) was the major lipid increased by ESK981 (FIG. 11b). As mentioned above, PE is an important membrane component for phagophores, autophagosomes, and autolysosomes, and increased intracellular PE levels can positively regulate autophagosome biogenesis⁴³. These results suggested that ESK981 may directly target a factor involved in cellular lipid metabolism to impact autophagy. To test whether ESK981 inhibited a lipid kinase, we performed an affinity binding screen for 22 phosphoinositide kinases and found that lipid kinase PIKfyve was the only target that ESK981 completely competed away relative to control (FIG. 11c). PIKfyve has previously been reported to maintain lysosome homeostasis; direct PIKfyve inhibition in mammalian cells, or homolog Fab1 in yeast, resulted in a vacuolization morphology and decreased autophagic flux^{13,44-46}. The binding potency was determined for ESK981 against several lipid kinases. The dissociation constant (Kd) for ESK981 against PIKfyve was 12 nM, while those for PIP5K1C, PIP5K1A, and PIK3CA were 210 nM, 230 nM, and greater than 10 μM, respectively (FIG. 11d). In order to determine whether PIKfyve was responsible for ESK981-associated vacuolization, individual lipid kinases were knocked down using siRNA, and only PIKfyve knockdown generated a cellular vacuolization phenotype that resembled ESK981 treatment (FIG. 11e, FIG. 12a-b). Autophagosome levels were further measured by CYTO-ID® and indicated increased autophagosome content after PIKfyve knockdown at levels similar to those induced by ESK981 in DU145, PC3, LNCaP, and VCaP cells (FIG. 11f-g). The cellular target engagement of ESK981 on PIKfyve was also confirmed by a cellular thermal shift assay (CETSA) in VCaP cells, with the known PIKfyve inhibitor apilimod serving as a positive control (FIG. 11h). Experiments were conducted that further confirmed that knockdown of PIKfyve increased CXCL10 expression and enhanced interferon response in human prostate cancer cells (FIG. 11i). Accordingly, lipid kinase PIKfyve is identified as a direct target of ESK981 that affects cellular lipid metabolism, autophagic flux, and autophagosome/lysosome levels in prostate cancer cells, thereby increasing CXCL10 expression.

### Example V

This example demonstrates that genetic inhibition of Pikfyve potentiates the therapeutic effect of anti-PD-1 immunotherapy in syngeneic murine prostate cancer models.

PIKfyve has been reported as a therapeutic target in B cell non-Hodgkin's lymphoma, multiple myeloma, and autophagy-dependent cancers^{13,15,44}; however, its role in syngeneic models has not been well studied. To investigate whether inhibiton of PIKfyve is reponsible for anti-tumor effects and immune activation phenotypes observed with ESK981 treatment in prostate cancer, experiments were conducted that generated Myc-CaP cells with doxycycline-inducible Pikfyve knockdown (shPikfyve). Upon Pikfyve knockdown, Myc-CaP cells displayed a cellular vacuolization morphology resembling ESK981 treatment (FIG. 13a). Tumor proliferation was measured with shPikfyve Myc-CaP cells in both immune-competent (FVB) and immune-deficient mice (NSG) (FIG. 13b). Tumor proliferation and waterfall plots of individual mice showed that Pikfyve knockdown had greater tumor inhibitory effects in FVB mice than in NSG mice, suggesting a competent immune environment is required for maximizing Pikfyve inhibition-induced anti-tumor responses (FIG. 13c-f). Finally, the effects of Pikfyve knockdown in combination with anti-PD-1 were examined using the Myc-CaP model (FIG. 13g). Similar to ESK981 treatment (FIG. 7d), Pikfyve knockdown showed significant tumor inhibition effects in FVB mice, and combination treatment with anti-PD-1 further enhanced the tumor inhibitory effect (FIG. 13h). The combination of anti-PD-1 with Pikfyve knockdown significantly increased complete tumor regression, defined as the percent cure rate, of Myc-CaP tumors from 0 to 40% (FIG. 13i). These results demonstrate that the lipid kinase PIKfyve is a promising target in prostate cancer, and targeting PIKfyve sensitizes the therapeutic effect of anti-PD-1 in prostate cancer.

### Example VI

This example provides a discussion of the experiments described in Examples I-V.

The major significance of this study relies on identifying phase I-cleared compound ESK981 as a novel PIKfyve inhibitor that effectively blocks the progression of multiple models of advanced prostate cancer, and this anti-tumor effect can be further capitalized upon by combining with anti-PD-1 therapy. Experiments were conducted demonstrating that PIKfyve inhibition leads to upregulation of cellular autophagosome and lysosome levels with blocked autophagic flux. PIKfyve inhibition increases tumor cell expression and secretion of chemokine CXCL10 to recruit T cells into the tumor microenvironment, resulting in enhanced anti-tumor efficacy in prostate cancer. In sum, it was shown that PIKfyve inhibition converts prostate cancers from immune cold tumors to inflamed tumors with increased treatment susceptibility to immune checkpoint blockade (FIG. 13j).

Prostate cancers are poorly immune-infiltrated tumors, and immune checkpoint inhibitor monotherapy in unselected advanced prostate cancer patient populations has thus had minimal success. In two phase III trials, the CTLA4 inhibitor ipilimumab failed to improve overall survival, while the PD-1 inhibitor pembrolizumab had low response rates (3-5%) in men with metastatic CRPC (mCRPC)^{22,23,47}. In the 5-10% of advanced prostate cancers harboring mismatch repair deficiency (MMRd)/microsatellite instability (MSI) and the 5-7% of patients harboring CDK12 loss of function mutations, unregulated mutation-associated neoantigens render these tumors more susceptible to immunotherapy, yet only 50% response rates are still achieved⁴⁸⁻⁵¹. Therefore, there remains an urgent need to convert the majority of prostate cancers from immune cold tumors to inflamed hot tumors and render patients susceptible to immunotherapies. Identification of ESK981 as a novel PIKfyve inhibitor accelerates the clinical translation of these findings in treating advanced prostate cancer as a monotherapy and in combination with immune checkpoint inhibitor therapy. Based on such findings, phase II clinical trials of ESK981 alone (NCT03456804) or in combination with nivolumab (NCT04159896) in mCRPC have begun.

Autophagy is a complex cellular process whose role in cancer biology continues to be defined. The impact of autophagy blockade on the inhibition of tumor progression has been well documented by several studies such as those in pancreatic cancer, prostate cancer, and genetically modified murine models⁵²⁻⁵⁴. Elevated autophagy was reported to be a treatment resistance and survival mechanism for tumor progression; pharmacologically or genetically blocking autophagy impairs prostate cancer survival and overcomes enzalutamide resistance in CRPC, implying the therapeutic potential of autophagy inhibitors in the antiandrogen-resistant setting⁵³. However, the effect of targeting autophagy on the immune landscape of tumors is still only partially defined.

Several autophagy proteins have been recently identified as druggable targets in tumor progression. A recent study reported that autophagy inhibition by genetic or pharmacological inhibition of VPS34 recruits T cells and NK cells into the tumor microenvironment through the expression and secretion of chemokines CXCL10 and CCLS in multiple syngeneic murine models, resulting in enhanced therapeutic benefit of anti-PD-1/PD-L1 and reprogramming of a cold tumor to a hot inflamed tumor¹⁹. Additionally, genetic targeting of Beclin1 in melanoma cells induced a massive infiltration of NK cells into tumors²⁰. Moreover, systemic deletion of Atg7 in mice significantly reduced the tumor growth of melanoma models through degradation of arginine that is required for tumor growth⁵². A recent study has also suggested that autophagy inhibition may sensitize tumors to other immunomodulatory mechanisms, such as restoring cell surface expression of MHC-I²¹.

Through the molecular analysis of ESK981-induced vacuolization described herein, the interesting finding that autophagosome accumulation by ESK981 treatment was recapitulated in yeast systems was uncovered, indicating that ESK981 shared a common target in both species, even though eukaryotic-like tyrosine kinases are absent in yeast³⁴. A subsequent kinase screen revealed that ESK981 is a novel pharmacological inhibitor of the lipid kinase PIKfyve. Inhibition of PIKfyve by ESK981 in prostate cancer cells yielded a massive cellular vacuolization phenotype with increased autophagosome and lysosome accumulation. These findings are consistent with genetic inactivation of PIKfyve in mammalian cells or yeast orthologue Fab1 and an increased cellular vacuole morphology^{55,56}. Identification of PIKfyve as a target of ESK981 provides a direct mechanistic connection between the increased lipid metabolism by ESK981 and autophagosome and lysosome accumulation with blocked autophagic flux. Such findings are also consistent with previous reports on the effects of PIKfyve inhibition on autophagic flux^{44,57}.

Such experiments are the first to demonstrate that PIKfyve is a therapeutic target in advanced prostate cancer and that pharmacological inhibition of PIKfyve by ESK981 turns immunologically cold tumors to hot inflamed tumors. The identification of PIKfyve as the molecular target of ESK981 yields an additional target with the potential to be combined with immune checkpoint blockade for treatment of advanced prostate cancer. Overall, pharmacological or genetic inactivation of PIKfyve in prostate cancer cells increases chemokine CXCL10 expression and secretion, thereby promoting increased cellular response to interferon γ stimulation. It was further shown that CXCL10 secretion is mediated through ATG5, suggesting that tumor cell autophagosome formation capability is functionally important for chemokine secretion and crosstalk with immune cells in the tumor microenvironment. In accordance with such findings, PIKfyve inhibition with the small molecule apilimod has shown tumor inhibitory effects in B cell non-Hodgkin's lymphoma and potentiation of anti-PD-L1 anti-tumor effects in a syngeneic model of lymphoma with A20 cells¹³.

Overcoming resistance to immune checkpoint blockade-based therapies and increasing their objective response rates are still unmet clinical needs and have become urgent challenges. The experiments described herein provide the first proof of concept to design innovative and rational clinical trials using PIKfyve inhibition in combination with immune checkpoint blockade. Such combination therapy will likely extend the benefit of cancer immune therapies to initial non-responder patients.

### Example VII

This example describes the materials and methods utilized in implementing the experiments described in Examples I-V.

### Cell Culture

All cell lines were obtained from ATCC, unless otherwise stated. VCaP cells were maintained in DMEM with Glutamax (Gibco). LNCaP, 22RV1, C4-2B, LNCaP-AR, PC3, and DU145 cells were maintained in RPMI 1640. Enzalutamide-resistant LNCaP-AR and CWR-R1 cells were grown in RPMI 1640 supplemented with 5 μM or 20 μM enzalutamide, respectively. Myc-CaP mouse prostate cancer cells were maintained in DMEM with Glutamax. All cells were supplemented with 10% FBS (Invitrogen) and grown in 5% CO₂ cell culture incubators. MEF Atg5^{+/+} and Atg5^{-/-} cells were provided by RIKEN BioResource. The parental LNCaP-AR prostate cancer cell line was kindly provided by Charles Sawyers⁵⁸. Cell lines were regularly checked for mycoplasma and authenticated.

### Compounds

ESK981 was initially chemically synthesized by K.D. Subsequently, ESK981 was provided by Esanik Therapeutics which licensed the compound from Teva Pharmaceuticals. Tyrosine kinase inhibitor library (Cat No. L1800), autophagy compound library (Cat No. L2600), and other compounds were purchased from Selleckchem.

### Long-Term Survival Assay and IC₅₀ Calculation

Single cell suspensions were seeded into 96-well plates at a density of 1,000-30,000 cells per well. Long-term survival was determined after 14 days of drug incubation. Viable cells were fixed with 4% formaldehyde and subsequently stained with 1% crystal violet. IC₅₀ was calculated using GraphPad Prism.

### Autophagy Detection for Compound Screening

10,000 cells were plated in 96-well plates and incubated with 300 nM of the various compounds for 24 hours. Autophagy activities were detected with CYTO-ID® Autophagy detection kit (Cat No. ENZ-KIT175, Enzo Life Science) according to the manufacturer's instructions, with fluorescence intensity measured on a TECAN M1000 plate reader. Autophagosome induction factor was calculated according to the manufacturer's instructions.

### Autophagic Flux Detection

GFP-LC3-RFP-LC3AG expressing PC3 and DU145 cells were stably transfected with pMRX-IP-GFP-LC3-RFP-LC3AG plasmid (Addgene #84572), and single cell clones were validated to avoid homologous recombination between the two LC3 fragments during retrovirus infection. For autophagic flux detection, 10,000 cells were plated in 96-well plates and incubated with various compounds in complete medium for 24 hours. GFP and RFP fluorescence intensities were measured on a TECAN M1000 plate reader.

### RNA In Situ Hybridization (ISH)

The RNAscope 2.5 HD BROWN Assay (Cat No. 322300; Advanced Cell Diagnostics) was performed according to the manufacturer's instructions and used target probes on whole tissue sections. Cd3 RNA probes (Cat No. 314721, Advanced Cell Diagnostics) and Cxcl10 RNA probes (Cat No. 408921, Advanced Cell Diagnostics) were complementary to the target mRNA. Probes Mm-PPIB (mouse peptidylprolyl isomerase B) and DapB (bacterial dihydrodipicolinate reductase) were used as positive and negative controls, respectively. FFPE sections were baked at 60° C. for 1 hour. Tissues were first deparaffinized by immersing in xylene twice for 5 minutes each with periodic agitation. The slides were then immersed in 100% ethanol twice for 1 minute each with periodic agitation and then air-dried for 5 minutes. Following a series of pretreatment steps, the cells were permeabilized using Protease Plus to enable probe access to the RNA targets. Post hybridization (HybEZ Oven for 2 hours at 40° C.), slides were washed twice and processed for standard signal amplification steps. Chromogenic detection was performed using DAB, followed by counterstaining with 50% Gill's Hematoxylin I (26801-01, Fisher Scientific). The RNA ISH signal was identified as brown, punctate dots.

### Immunohistochemistry

Immunohistochemistry (IHC) was performed on formalin-fixed paraffin-embedded tumor tissue sections, using anti-Ki-67 rabbit monoclonal primary antibody (Cat No. 790-4286, Ventana Medical Systems). IHC was carried out using an automated protocol developed for the Benchmark XT automated slide staining system (Ventana Medical Systems) and was detected using ultraView Universal DAB detection kit (Cat No. 760-500, Ventana Medical Systems). Hematoxylin II (Cat No. 790-2208, Ventana-Roche) was used as counterstain.

### Cytokine Array

Cells were seeded in 6-well plates, and conditioned medium was collected after 24 hours of drug incubation. Cytokine expression was determined by proteome profiler mouse XL cytokine array (Cat No. ARY028, R&D System) or proteome profiler human XL cytokine array kit (Cat No. ARY022, R&D System) according to the manufacturer's instructions.

### ELISA

Conditioned medium was collected after 24 hours of drug incubation. ELISA was performed using a human CXCL10 ELISA kit (Cat No. KAC2361, ThermoFisher), or a mouse CXCL10 ELISA kit (Cat No. ab214563, Abcam) according to the manufacturer's instructions.

### Cell Cycle Analysis

Cells were seeded in 6-well plates and treated with various drugs for 72 hours. Single cells were fixed with 70% ethanol, stained with propidium iodine, and cell cycle was analyzed by flow cytometry.

### Matrigel Invasion Assay

2-5×10⁵ cells were seeded onto 8-μm Matrigel-coated fluoroblok transwells with serum-free medium and various concentrations of ESK981. Medium containing 10% FBS in the lower chamber served as a chemoattractant. After 24-hour incubation, invaded cells were stained with calcium AM green at 37° C., and fluorescence intensity was quantified with a TECAN M1000 plate reader.

### 3D Spheroids

Nuclear red fluorescent protein-expressing VCaP cells were seeded in ultralow attachment 96-well plates and spun down at 1000 rpm for 10 minutes to pellet cells. Spheroids were formed after 3 days of incubation in a cell culture incubator, and then treatment was started. Red fluorescence intensity was monitored by IncuCyte ZOOM.

### Yeast Autophagy Detection

The yeast strains used in this study were YAB499 (SEY6210, pho13Δ pho8Δ60, pdr5Δ::Kan). Protein extraction and immunoblot were performed as previously described⁵⁹. Antisera to Atg8 and Pgk1 (a generous gift from Dr. Jeremy Thorner, University of California, Berkeley) were used as previously described. Cells were treated with either 3 μM ESK981, 3 μM cabozantinib, or the equivalent amount of DMSO as control for the indicated times.

### Immunoblotting, Immunofluorescence, and Antibodies

Cell lysates were harvested in Pierce RIPA Lysis buffer (Thermo Scientific) containing protease inhibitor cocktail tablets (Roche) and phosphatase inhibitor cocktail (Millipore). Protein concentration was measured using the DC Protein Assay (Bio-Rad) to ensure an equal amount of protein was loaded onto a gel. The denatured lysates were separated on NuPage 4-12% Bis-Tris Midi Protein gels (Novex) and transferred to 0.45-μm PVDF transfer membrane (Immobilon) using a TransBlot Turbo dry transfer machine (Bio-Rad). The membrane was incubated in blocking buffer (5% non-fat dry milk, Tris-buffered saline with 0.1% Tween 20) for 1 hour at room temperature. The membrane was then incubated with the listed antibodies for 1 hour at room temperature, followed by overnight incubation at 4° C. Chemiluminescent detection using ECL Prime (Amersham) and HyBlot CL autoradiography film (Denville Scientific) was used to visualize the blots. Antibodies used in the immunoblotting assays were against ATG5 (Cat No. 12994S, Cell Signaling Technology), ATG7 (Cat No. 8558S, Cell Signaling Technology), ULK1 (Cat No. 8054S, Cell Signaling Technology), Beclin1 (Cat No. 4122S, Cell Signaling Technology), FIP200 (Cat No. 12436S, Cell Signaling Technology), LC3A/B (Cat No. 127415, Cell Signaling Technology), PIKfyve (Cat No. AF7885, R&D Systems), and GAPDH (Cat No. 3683S, Cell Signaling Technology). All antibodies were used at dilutions suggested by the manufacturers.

For LAMP1 immunofluorescence, cells were seeded on coverslips overnight and treated with ESK981 at 300 nM for 24 hours. Coverslips were fixed with 10% paraformaldehyde and permeabilized with 10% saponin. Coverslips were then blocked with 10% goat serum and stained with LAMP1 antibody (Cat No. 9091, Cell Signaling Technology) and fluorescently-labelled secondary antibody. Confocal images were taken using a Nikon A1 confocal microscope.

For GFP-LC3 confocal imaging, GFP-LC3 expressing DU145 cells were seeded on coverslips overnight and treated with ESK981 at 300 nM for various time points. Coverslips were then fixed with 10% paraformaldehyde. Confocal images were taken using a Nikon A1 confocal microscope.

### RNA Isolation and Quantitative Real-Time PCR

Total RNA was extracted from cells or tissue using the miRNeasy mini kit (Qiagen), and cDNA was synthesized from 1 μg total RNA using the High Capacity cDNA Reverse Transcription Kit (Applied Biosystems). qPCR was performed using Fast SYBR Green Master Mix (Applied Biosystems) on the ViiA7 Real-Time PCR System (Applied Biosystems). The target mRNA expression was quantified using the ΔΔCt method and normalized to GAPDH expression. The primer sequences used for the SYBR green qPCR are as follows:

GAPDH qPCR forward,
(SEQ ID NO: 1)
TGCACCACCAACTGCTTAGC;
GAPDH qPCR reverse,
(SEQ ID NO: 2)
GGCATGGACTGTGGTCATGAG;
CXCL10 qPCR forward,
(SEQ ID NO: 3)
GGTGAGAAGAGATGTCTGAATCC;
CXCL10 qPCR reverse,
(SEQ ID NO: 4)
GTCCATCCTTGGAAGCACTGCA;
CXCL9 qPCR forward,
(SEQ ID NO: 5)
CTGTTCCTGCATCAGCACCAAC;
CXCL9 qPCR reverse,
(SEQ ID NO: 6)
TGAACTCCATTCTTCAGTGTAGCA;
PIKFYVE qPCR forward:
(SEQ ID NO: 7)
CTGAGTGATGCTGTGTGGTCAAC;
PIKFYVE qPCR reverse:
(SEQ ID NO: 8)
CAAGGACTGACACAGGCACTAG;
PIP5K1C qPCR forward:
(SEQ ID NO: 9)
ACTACAGCCTCCATTGCCACGA;
PIP5K1C qPCR reverse:
(SEQ ID NO: 10)
CATCCTGTCCAGACGACTGTGT;
PIK3CA qPCR forward:
(SEQ ID NO: 11)
GAAGCACCTGAATAGGCAAGTCG;
PIK3CA qPCR reverse:
(SEQ ID NO: 12)
GAGCATCCATGAAATCTGGTCGC;
Gapdh qPCR forward,
(SEQ ID NO: 13)
CATCACTGCCACCCAGAAGACTG;
Gapdh qPCR reverse,
(SEQ ID NO: 14)
ATGCCAGTGAGCTTCCCGTTCAG;
Cxcl10 qPCR forward,
(SEQ ID NO: 15)
ATCATCCCTGCGAGCCTATCCT;
Cxcl10 qPCR reverse,
(SEQ ID NO: 16)
GACCTTTTTTGGCTAAACGCTTTC;
Cxcl9 qPCR forward,
(SEQ ID NO: 17)
CCTAGTGATAAGGAATGCACGATG;
Cxcl9 qPCR reverse,
(SEQ ID NO: 18)
CTAGGCAGGTTTGATCTCCGTTC;
Cd3e qPCR forward,
(SEQ ID NO: 19)
GCTCCAGGATTTCTCGGAAGTC;
Cd3e qPCR reverse,
(SEQ ID NO: 20)
ATGGCTACTGCTGTCAGGTCCA.

### RNA Interference and Short Hairpin RNA

For transient knockdown experiments, cells were seeded in 6-well plates and transfected with 100 nM ON-TARGETplus SMARTpool siRNA (Thermo Scientific) targeting PIKFYVE (ON-TARGETplus Human PIKFYVE_SMARTpool, catalog no. L-005058-00-0005), PIP5K1C (ON-TARGETplus Human PIP5K1C_SMARTpool, catalog no. L-004782-00-0005), PIK3CA (ON-TARGETplus Human PIK3CA_SMARTpool, catalog no. L-003018-00-0005), or non-targeting control (Non-targeting Pool, catalog no. D-001810-10-50) using Lipofectamine® RNAiMAX (Invitrogen) according to the manufacturer's instructions. For stable doxycycline inducible shPIKfyve Myc-CaP cells, a SMARTvector lentiviral shRNA construct encoding a PIKfyve targeting sequence (TGGTGTCTGCGCCTAAATG (SEQ ID NO: 21)) was used to infect Myc-CaP cells, and positively-infected cells were selected by puromycin.

### LysoTracker Green Flow Cytometry Analysis

Cells were grown in 6-well plates and treated with various drugs. After 24 hours, cells were stained with LysoTracker® Green DND-26 (Invitrogen), and green fluorescence signal was analyzed by flow cytometry.

### Cellular Thermal Shift Assay

The ability of compounds to interact with, and thereby stabilize the target in intact cells, was analyzed essentially as previously described^{60,61}. The target engagement of ESK981 to PIKfyve was performed in VCaP cells. Cells were treated with DMSO, ESK981 (1 μM), or apilimod (1 μM) for 2 hours at 37° C. and 5% CO₂, and 1×10⁶ single cell suspensions were diluted into 50 μl of PBS containing protease inhibitor. Cell suspensions were then incubated in a PCR thermal cycler at various temperatures for 2 cycles of 3 minutes heating followed by 3 minutes cooling at room temperature. Cells were lysed by three cycles of freeze-thawing using liquid nitrogen. 20 μl of the soluble fraction of cell lysates were analyzed by western blot.

### Murine Prostate Tumor Xenograft Models

Four- to six-week old male CB17 severe combined immunodeficiency (SCID) mice were procured from the University of Michigan breeding colony. Subcutaneous tumors were established at both sides of the dorsal flank of mice. Tumors were measured at least biweekly using digital calipers following the formula (π/6) (L×W2), where L=length and W=width of the tumor. At the end of the studies, mice were sacrificed and tumors extracted and weighed. The University of Michigan University Committee on the Use and Care of Animals (UCUCA) approved all in vivo studies. For the VCaP castration-resistant tumor model, 3×10⁶ VCaP cells were injected subcutaneously into the dorsal flank on both sides of the mice in serum-free medium with 50% Matrigel (BD Biosciences). Once tumors reached a palpable stage (˜200 mm³), tumor-bearing mice were castrated. Once tumors grew back to the pre-castration size, mice were randomized and treated with either 30 mg/kg, 60 mg/kg ESK981, or vehicle (ORA-PLUS) by oral gavage 5 days per week. For the DU145 xenograft tumor model, 1×10⁶ DU145 cells were injected subcutaneously into the dorsal flank on both sides of the mice in serum-free medium with 50% Matrigel. When tumors reached ˜100 mm³, tumor-bearing mice were randomized and treated with 30 mg/kg ESK981 or vehicle (ORA-PLUS) by oral gavage 5 days per week.

### Prostate Patient-Derived Xenograft Models

The University of Texas M.D. Anderson Cancer Center (MDACC) patient-derived xenografts (PDX) series has been previously described⁶²⁻⁶⁴. PDXs were derived from men with CRPC undergoing palliative resections using described protocols^{65,66}. MDA-PCa-146-12 and 146-10 PDX were derived from a patient initially diagnosed with Gleason 3+4=7 prostate adenocarcinoma with an initial PSA of 10.7 ng/ml. Histopathological evaluation of the cystoprostatectomy specimen demonstrated mixed prostatic adenocarcinoma and small cell carcinoma involving the prostate, seminal vesicles, and urinary bladder wall. MDA-PCa-146-12 was derived from a specimen obtained from the left bladder wall and demonstrated conventional adenocarcinoma, while MDA-PCa-146-10 was derived from the bladder wall and had small cell carcinoma morphology. PDXs were maintained in male SCID mice by surgically implanting 2 mm³ tumors coated with 100% Matrigel to both flanks of mice. Once tumors reached 100-200 mm³ in size, mice were randomized and divided into different treatment groups receiving either 30 mg/kg ESK981 or vehicle (ORA-PLUS) by oral gavage 5 days per week.

### Syngeneic Murine Prostate Models

MYC-driven murine prostate cancer cells (Myc-CaP) were injected at a density of 1×10⁶ subcutaneously into both flanks of 4- to 6-week old FVB mice (Charles River Laboratories) in serum-free medium with 50% Matrigel. When tumors reached ˜50 mm³, tumor-bearing mice were randomized and treated with 15 mg/kg or 30 mg/kg ESK981 or vehicle (ORA-PLUS) by oral gavage 5 days per week. For the ESK981 and anti-PD-1 combination study, 15 mg/kg ESK981 or vehicle were given 5 days per week by oral gavage, while anti-PD-1 (Cat No. BE0146, BioXcell) or isotype control (Cat No. BE0089, BioXcell) were given at 200 μg per mouse 3 times per week by intraperitoneal injection (i.p.). For the ESK981 and anti-CXCR3 combination study, 15 mg/kg ESK981 or vehicle were given 5 days per week by oral gavage, while anti-CXCR3 (Cat No. BE0249, BioXcell) or isotype control (Cat No. BE0091, BioXcell) were given at 100 μg per mouse 3 times per week by i.p. For the shPikfyve study, Myc-CaP shPikfyve cells were injected at a density of 2×10⁶ subcutaneously into both flanks of 4- to 6-week old NSG or FVB mice in serum-free medium with 50% Matrigel. When tumors reached 100 mm³, tumor-bearing mice were randomized and treated with normal diet or doxycycline 625 mg/kg diet (Envigo). For the shPikfyve and anti-PD-1 combination study, Myc-CaP shPikfyve cells were injected at a density of 2×10⁶ subcutaneously into both flanks of 4- to 6-week old FVB mice in serum-free medium with 50% Matrigel. When tumors reached 100 mm³, tumor-bearing mice were randomized and treated with normal diet or doxycycline 625 mg/kg diet for 7 days, and then anti-PD-1 or isotype control were given at 200 μg per mouse 3 times per week in combination with normal or doxycycline diet.

### Mouse Blood Chemistry

Whole blood was collected using BD Microtainer SST tubes, and serum was isolated by centrifugation at 7000 rpm for 10 minutes. Sera were submitted to the University of Michigan ULAM Pathology Cores for Animal Research for liver and renal chemistry analysis.

### Transmission Electron Microscopy (TEM)

Cells or fresh tissue were fixed with 2.5% glutaraldehyde in 0.1 M phosphate buffer, pH 7.4 for 24 hours at 4° C. and then rinsed twice with 0.1 M phosphate buffer. The University of Michigan Microscopy & Imaging core carried out sample embedding and imaging.

### CRISPR Atg5 Knockout Myc-CaP Cells

Guide RNAs (sgRNAs) targeting the exons of mouse Atg5 were designed using the CRISPR Design tool (crispr.mit.edu, F. Zhang laboratory, MIT). Non-targeting sgRNA (sgNT) (forward: ACGTGGGGACATATACGTGT (SEQ ID NO: 22); reverse: ACACGTATATGTCCCCACGT (SEQ ID NO: 23)) or Atg5 targeting sgRNA (sgAtg5) (forward: AAGAGTCAGCTATTTGACGT (SEQ ID NO: 24); reverse: ACGTCAAATAGCTGACTCTT (SEQ ID NO: 25)) were cloned into lentiCRISPR v2 plasmid according to published literature⁶⁷. lentiCRISPR v2 plasmid was a gift from Feng Zhang (Addgene, #52961). Myc-CaP cells were transiently transfected with the sgNT or sgAtg5 plasmids. Post-transfection (72 hours), cells were subjected to puromycin selection for one week. Puromycin-resistant cells were resuspended into single cells and seeded into 96-well plates. One month later, Atg5 knockout (KO) clones were screened by western blot.

### Gene Expression Analysis

RNA was extracted from cell lines or tissue using the QIAGEN RNA extraction kit. RNA quality was determined by the Bioanalyzer RNA Nano Chip. Myc-CaP xenograft tumors used the RiboErase selection kit (Cat No. KK8561, Kapa Biosystems), while the remaining samples used the poly-A selection by Sera-Mag™ Oligo(dT)-Coated Magnetic Particles (Cat No. 38152103010150, GE Healthcare Life Sciences), and libraries were generated by KAPA RNA HyperPrep Kit (Cat No. KK8541, Roche Sequencing Solutions). RNA-sequencing was performed on the Illumina HiSeg™ 2500 platform. Differentially expressed genes and heatmaps were analyzed and drawn by Qlucore Omics analysis software. Gene Ontology analysis was performed at www.geneontology.org.

### Lipidomics

Details of sample preparation and identification for untargeted lipidomic profiling have been previously reported⁶⁸. The lipids were extracted using a modified Bligh-Dyer method⁶⁹. The extraction was carried out using a 2:2:2 volume ratio of water:methanol:dichloromethane at room temperature after spiking internal standard lipids (17:0LPC, 17:0PC, 17:0PE, 17:0PG, 17:0 ceramide, 17:0SM, 17:0PS, 17:0PA, 17:0TG, 17:0MG, d5-DG, d31-TG, and 17.0-20.4 PI). The organic layer was collected and dried completely under nitrogen. The organic dried extract containing lipids was further analyzed by LC-MS-based lipidomics. The dried lipid extracts were injected onto a 1.8-μm particle 50×2.1 mm id Waters Acquity HSS T3 column (Waters, Milford, MA), which was heated to 55° C. A binary gradient system consisting of acetonitrile and water with 10 mM ammonium acetate (40:60, v:v) was used as eluent A. Eluent B consisted of water, acetonitrile, and isopropanol, both containing 10 mM ammonium acetate (510:85, v:v). The lipid extracts were reconstituted with a buffer B and injected to MS. The MS analysis alternated between MS and data-dependent MS2 scans using dynamic exclusion in both positive and negative polarity. As controls (QC) to monitor the profiling process, a pool of plasma and test plasma (a small aliquot from all test samples) were extracted and analyzed in tandem with the experimental samples. These controls were incorporated multiple times into the randomization scheme such that sample preparation and analytical variability could be constantly monitored. Lipids were identified using LIPIDBLAST library⁷⁰ (computer-generated tandem mass spectral library of 212,516 spectra covering 119,200 compounds from 26 lipid compound classes, including phospholipids, glycerolipids, bacterial lipoglycans, and plant glycolipids) by matching the product ions MS/MS data. Mass spectrometry data files were processed using MultiQuant 1.1.0.26 (Applied Biosystems/MDS Analytical Technologies). Identified lipids were quantified by normalizing against their respective internal standard. QC samples were used to monitor the overall quality of the lipid extraction and mass spectrometry analyses. The QC samples were mainly used to remove technical outliers and lipid species that were detected below the lipid class-based lower limit of quantification.

### Lipid Kinase Competition Assay

Lipid kinase competition assays for 22 lipid kinases, including clinically-relevant mutants, were performed using DiscoveRX KINOEscan® platform scanLIPID® panel. Detailed information is described on the DiscoveRX website (https://www.discoverx.com/technologies-platforms/competitive-binding-technology/kinomescan-technology-platform).

### Kinase Dissociation Constant Analysis

Quantitative binding constants (Kd) of ESK981 to PIKfyve, PIP5K1A, PIP5K1C, and PIK3CA were generated using KdELECT® platform (DiscoveRX). An 11-point dose-response of ESK981 (0.05-3000 nM) was used, and the experiment was performed in duplicate. Detailed information is described on the DiscoveRX website (www.discoverx.com).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes. Specifically, the following references denoted herein are incorporated by reference for all purposes:
1. Siegel, R. L., Miller, K.D. & Jemal, A. Cancer statistics, 2020. CA Cancer J Clin 70, 7-30 (2020).
2. Ferraldeschi, R., Welti, J., Luo, J., Attard, G. & de Bono, J. S. Targeting the androgen receptor pathway in castration-resistant prostate cancer: progresses and prospects. Oncogene 34, 1745-1757 (2015).
3. Scher, H. I., et al. Increased survival with enzalutamide in prostate cancer after chemotherapy. N. Engl. J. Med. 367, 1187-1197 (2012).
4. de Bono, J. S., et al. Abiraterone and increased survival in metastatic prostate cancer. N. Engl. J. Med. 364, 1995-2005 (2011).
5. Qiao, Y., et al. Mechanistic support for combined MET and AR blockade in castration-resistant prostate cancer. Neoplasia 18, 1-9 (2016).
6. Ahronian, L. G. & Corcoran, R. B. Strategies for monitoring and combating resistance to combination kinase inhibitors for cancer therapy. Genome Med. 9, 1-12 (2017).
7. Smith, D. C., et al. Cabozantinib in patients with advanced prostate cancer: results of a phase II randomized discontinuation trial. J. Clin. Oncol. 31, 412-419 (2013).
8. Smith, M., et al. Phase III study of cabozantinib in previously treated metastatic castration-resistant prostate cancer: COMET-1. J. Clin. Oncol. 34, 3005-3013 (2016).
9. Hudkins, R. L., et al. Synthesis and biological profile of the pan-vascular endothelial growth factor receptor/tyrosine kinase with immunoglobulin and epidermal growth factor-like homology domains 2 (VEGF-R/TIE-2) inhibitor 11-(2-methylpropyl)-12,13-dihydro-2-methyl-8-(pyrimidin-2-ylamino)-4H-indazolo[5, 4-a]pyrrolo[3,4-c]carbazol-4-one (CEP-11981): a novel oncology therapeutic agent. J. Med. Chem. 55, 903-913 (2012).
10. Eroglu, Z., Stein, C. A. & Pal, S. K. Targeting angiopoietin-2 signaling in cancer therapy. Expert Opin. Investig. Drugs 22, 813-825 (2013).
11. Pili, R., Carducci, M., Brown, P. & Hurwitz, H. An open-label study to determine the maximum tolerated dose of the multitargeted tyrosine kinase inhibitor CEP-11981 in patients with advanced cancer. Invest. New Drugs 32, 1258-1268 (2014).
12. Shisheva, A. PIKfyve: Partners, significance, debates and paradoxes. Cell Biol. Int 32, 591-604 (2008).
13. Gayle, S., et al. Identification of apilimod as a first-in-class PIKfyve kinase inhibitor for treatment of B-cell non-Hodgkin lymphoma. Blood 129, 1768-1778 (2017).
14. Baird, A. M., et al. IL-23R is epigenetically regulated and modulated by chemotherapy in non-small cell lung cancer. Front. Oncol. 3, 162 (2013).
15. Bonolo De Campos, C., et al. Identification of PIKfyve kinase as a target in multiple myeloma. Haematologica 105, 1641-1649 (2019).
16. Levy, J. M. M., Towers, C. G. & Thorburn, A. Targeting autophagy in cancer. Nat. Rev. Cancer 17, 528-542 (2017).
17. Kroemer, G., Galluzzi, L., Kepp, 0. & Zitvogel, L. Immunogenic cell death in cancer therapy. Annu. Rev. Immunol. 31, 51-72 (2013).
18. Michaud, M., et al. Autophagy-dependent anticancer immune responses induced by chemotherapeutic agents in mice. Science 334, 1573-1577 (2011).
19. Noman, M. Z., et al. Inhibition of Vps34 reprograms cold into hot inflamed tumors and improves anti-PD-1/PD-L1 immunotherapy. Sci. Adv. 6, eaax7881 (2020).
20. Mgrditchian, T., et al. Targeting autophagy inhibits melanoma growth by enhancing NK cells infiltration in a CCLS-dependent manner. Proc. Natl. Acad. Sci. U.S.A. 114, E9271-E9279 (2017).
21. Yamamoto, K., et al. Autophagy promotes immune evasion of pancreatic cancer by degrading MHC-I. Nature 581, 100-105 (2020).
22. Beer, T. M., et al. Randomized, double-blind, phase III trial of ipilimumab versus placebo in asymptomatic or minimally symptomatic patients with metastatic chemotherapy-naive castration-resistant prostate cancer. J Clin. Oncol. 35, 40-47 (2017).
23. Kwon, E. D., et al. Ipilimumab versus placebo after radiotherapy in patients with metastatic castration-resistant prostate cancer that had progressed after docetaxel chemotherapy (CA184-043): a multicentre, randomised, double-blind, phase 3 trial. Lancet Oncol. 15, 700-712 (2014).
24. Wheeler, D. L., Iida, M. & Dunn, E. F. The role of Src in solid tumors. Oncologist 14, 667-678 (2009).
25. Nagasawa, J., et al. Novel HER2 selective tyrosine kinase inhibitor, TAK-165, inhibits bladder, kidney and androgen-independent prostate cancer in vitro and in vivo. Int. J. Urol. 13, 587-592 (2006).
26. Harshman, L. C., et al. An investigator-initiated phase I study of crizotinib in combination with enzalutamide in metastatic castration-resistant prostate cancer (mCRPC) before or after progression on docetaxel. J. Clin. Oncol. 34, e16509 (2016).
27. Hickman, J. A., et al. Three-dimensional models of cancer for pharmacology and cancer cell biology: capturing tumor complexity in vitro/ex vivo. Biotechnol. J. 9, 1115-1128 (2014).
28. Harma, V., et al. A comprehensive panel of three-dimensional models for studies of prostate cancer growth, invasion and drug responses. PloS one 5, e10431 (2010).
29. Robinson, D., et al. Integrative clinical genomics of advanced prostate cancer. Cell 161, 1215-1228 (2015).
30. Yang, Z. J., Chee, C. E., Huang, S. & Sinicrope, F. A. The role of autophagy in cancer: therapeutic implications. Mol. Cancer Ther. 10, 1533-1541 (2011).
31. Klionsky, D. J., et al. Guidelines for the use and interpretation of assays for monitoring autophagy (3rd edition). Autophagy 12, 1-222 (2016).
32. Marx, V. Autophagy: eat thyself, sustain thyself. Nat. Methods 12, 1121-1125 (2015).
33. Kim, J. & Klionsky, D. J. Autophagy, cytoplasm-to-vacuole targeting pathway, and pexophagy in yeast and mammalian cells. Annu. Rev. Biochem. 69, 303-342 (2000).
34. Miller, W. T. Tyrosine kinase signaling and the emergence of multicellularity. Biochim. Biophys. Acta. 1823, 1053-1057 (2012).
35. Kaizuka, T., et al. An autophagic flux probe that releases an internal control. Mol. Cell 64, 835-849 (2016).
36. Kraya, A. A., et al. Identification of secreted proteins that reflect autophagy dynamics within tumor cells. Autophagy 11, 60-74 (2015).
37. Liu, M., Guo, S. & Stiles, J. K. The emerging role of CXCL10 in cancer. Oncol. Lett. 2, 583-589 (2011).
38. Harlin, H., et al. Chemokine expression in melanoma metastases associated with CD8+ T-cell recruitment. Cancer Res. 69, 3077-3085 (2009).
39. Bronger, H., et al. CXCL9 and CXCL10 predict survival and are regulated by cyclooxygenase inhibition in advanced serous ovarian cancer. Br. J. Cancer 115, 553-563 (2016).
40. Tokunaga, R., et al. CXCL9, CXCL10, CXCL11/CXCR3 axis for immune activation a target for novel cancer therapy. Cancer Treat. Rev. 63, 40-47 (2018).
41. Watson, P. A., et al. Context-dependent hormone-refractory progression revealed through characterization of a novel murine prostate cancer cell line. Cancer Res. 65, 11565-11571 (2005).
42. Serganova, I., et al. Enhancement of PSMA-directed CAR adoptive immunotherapy by PD-1/PD-L1 blockade. Mol. Ther. Oncolytics 4, 41-54 (2017).
43. Rockenfeller, P., et al. Phosphatidylethanolamine positively regulates autophagy and longevity. Cell Death Differ. 22, 499-508 (2015).
44. Sharma, G., et al. A family of PIKFYVE inhibitors with therapeutic potential against autophagy-dependent cancer cells disrupt multiple events in lysosome homeostasis. Autophagy 15, 1694-1718 (2019).
45. Gayle, S., et al. B-cell non-Hodgkin lymphoma: Selective vulnerability to PIKFYVE inhibition. Autophagy 13, 1082-1083 (2017).
46. Efe, J. A., Botelho, R. J. & Emr, S. D. The Fab1 phosphatidylinositol kinase pathway in the regulation of vacuole morphology. Curr. Opin. Cell Biol. 17, 402-408 (2005).
47. Antonarakis, E. S., et al. Pembrolizumab for Treatment-Refractory Metastatic Castration-Resistant Prostate Cancer: Multicohort, Open-Label Phase II KEYNOTE-199 Study. J Clin Oncol 38, 395-405 (2020).
48. Abida, W., et al. Analysis of the Prevalence of Microsatellite Instability in Prostate Cancer and Response to Immune Checkpoint Blockade. JAMA Oncol 5, 471-478 (2019).
49. Antonarakis, E. S., et al. Clinical Features and Therapeutic Outcomes in Men with Advanced Prostate Cancer and DNA Mismatch Repair Gene Mutations. Eur Urol 75, 378-382 (2019).
50. Antonarakis, E. S., et al. CDK12-Altered Prostate Cancer: Clinical Features and Therapeutic Outcomes to Standard Systemic Therapies, Poly (ADP-Ribose) Polymerase Inhibitors, and PD-1 Inhibitors. JCO Precis Oncol 4, 370-381 (2020).
51. Wu, Y. M., et al. Inactivation of CDK12 delineates a distinct immunogenic class of advanced prostate cancer. Cell 173, 1770-1782 e1714 (2018).
52. Poillet-Perez, L., et al. Autophagy maintains tumour growth through circulating arginine. Nature 563, 569-573 (2018).
53. Nguyen, H. G., et al. Targeting autophagy overcomes enzalutamide resistance in castration-resistant prostate cancer cells and improves therapeutic response in a xenograft model. Oncogene 33, 4521-4530 (2014).
54. Yang, S., et al. Pancreatic cancers require autophagy for tumor growth. Genes Dev. 25, 717-729 (2011).
55. Sbrissa, D., Ikonomov, O. C. & Shisheva, A. PIKfyve, a mammalian ortholog of yeast Fablp lipid kinase, synthesizes 5-phosphoinositides. Effect of insulin. J Biol. Chem. 274, 21589-21597 (1999).
56. Jefferies, H. B., et al. A selective PIKfyve inhibitor blocks Ptdlns(3,5)P(2) production and disrupts endomembrane transport and retroviral budding. EMBO Rep. 9, 164-170 (2008).
57. Choy, C. H., et al. Lysosome enlargement during inhibition of the lipid kinase PIKfyve proceeds through lysosome coalescence. J. Cell Sci. 131(2018).
58. Chen, C. D., et al. Molecular determinants of resistance to antiandrogen therapy. Nat. Med. 10, 33-39 (2004).
59. Bernard, A., et al. Rph1/KDM4 mediates nutrient-limitation signaling that leads to the transcriptional induction of autophagy. Curr. Biol. 25, 546-555 (2015).
60. Martinez Molina, D., et al. Monitoring drug target engagement in cells and tissues using the cellular thermal shift assay. Science 341, 84-87 (2013).
61. Jafari, R., et al. The cellular thermal shift assay for evaluating drug target interactions in cells. Nat. Protoc. 9, 2100-2122 (2014).
62. Palanisamy, N., et al. Xenografts of human prostate cancer—a genetic profile analysis. Cancer Res. 73, 2780 (2013).
63. Tzelepi, V., et al. Modeling a lethal prostate cancer variant with small-cell carcinoma features. Clin. Cancer Res. 18, 666-677 (2012).
64. Aparicio, A., et al. Neuroendocrine prostate cancer xenografts with large-cell and small-cell features derived from a single patient's tumor: morphological, immunohistochemical, and gene expression profiles. The Prostate 71, 846-856 (2011).
65. Tomlins, S. A., et al. Role of the TMPRSS2-ERG gene fusion in prostate cancer. Neoplasia 10, 177-188 (2008).
66. Navone, N. M., Olive, M. & Troncoso, P. Isolation and culture of prostate cancer cell lines. Methods Mol. Med. 88, 121-132 (2004).
67. Sanjana, N. E., Shalem, 0. & Zhang, F. Improved vectors and genome-wide libraries for CRISPR screening. Nat. Methods 11, 783-784 (2014).
68. Afshinnia, F., et al. Lipidomic signature of progression of chronic kidney disease in the chronic renal insufficiency cohort. Kidney Int. Rep. 1, 256-268 (2016).
69. Bligh, E. G. & Dyer, W. J. A rapid method of total lipid extraction and purification. Can. J. Biochem. Physiol. 37, 911-917 (1959).
70. Cajka, T. & Fiehn, 0. LC-MS-based lipidomics and automated identification of lipids using the LipidBlast in-silico MS/MS library. Methods Mol. Biol. 1609, 149-170 (2017).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating, ameliorating, or preventing a hyperproliferative disease characterized with PIKfyve-expressing cells in a patient comprising administering to said patient a therapeutically effective amount of a composition comprising an agent capable of capable of inhibiting PIKfyve activity.

2. The method of claim 1, wherein the hyperproliferative disease is a cancer.

3. The method of claim 2, wherein the cancer is selected from prostate cancer, castration resistant prostate cancer, pancreatic cancer, colon cancer, melanoma, lung cancer, breast cancer, renal cancer, lymphoma, ovarian cancer, bladder cancer, Merkel cell carcinoma, rhabdomyosarcoma, osteosarcoma, synovial sarcoma, glioblastoma, Ewing's sarcoma, diffuse intrinsic pontine glioma (DIPG), neuroblastoma, and Wilms' tumor.

4. The method of claim 1, wherein the patient is a human patient.

5. The method of claim 1, wherein the agent capable of inhibiting PIKfyve activity is ESK981 (13-isobutyl-4-methyl-10-(pyrimidin-2-ylamino)-1,2,4,7,8,13-hexahydro-6H-indazolo[5,4-a]pyrrolo[3,4-c]carbazol-6-one) or a compound structurally similar to ESK981.

6. The method of claim 1, wherein the agent capable of inhibiting PIKfyve activity is further capable of one or more of the following: inhibiting conversion of phosphatidylinositol 3-phosphate (PI(3)P) to phosphatidylinositol 3,5-bisphosphate (PI(3,5)P₂), inhibiting PIKfyve activity related tumor growth, inhibiting PIKfyve activity related autophagic flux, and/or activating an anti-tumor immune response in cells having increased PIKfyve activity.

7. The method of claim 1, further comprising administering to said patient one or more anticancer agents, wherein said anticancer agent one or more of a chemotherapeutic agent, an immune checkpoint inhibitor, and radiation therapy.

8. The method of claim 6, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

9. The method of claim 7, wherein the PD-1 inhibitor is selected from nivolumab, pembrolizumab, STI-A1014, pidilzumab, and cemiplimab-rwlc.

10. The method of claim 7, wherein the PD-L1 inhibitor is selected from avelumab, atezolizumab, durvalumab, and BMS-936559.

11. The method of claim 7,
wherein the CTLA-4 inhibitor is selected from ipilimumab and tremelimumab,
wherein the LAG3 inhibitor is GSK2831781.

12. A kit comprising an agent capable of capable of inhibiting PIKfyve activity and instructions for administering said agent to a patient having a hyperproliferative disease characterized with PIKfyve-expressing cells.

13. The kit of claim 12, wherein the hyperproliferative disease is cancer, wherein the cancer is selected from prostate cancer, castration resistant prostate cancer, pancreatic cancer, colon cancer, melanoma, lung cancer, breast cancer, renal cancer, lymphoma, ovarian cancer, bladder cancer, Merkel cell carcinoma, rhabdomyosarcoma, osteosarcoma, synovial sarcoma, glioblastoma, Ewing's sarcoma, diffuse intrinsic pontine glioma (DIPG), neuroblastoma, and Wilms' tumor.

14. The kit of claim 12, further comprising one or more anticancer agents.

15. The kit of claim 12, wherein the anticancer agent is an immune checkpoint inhibitor.

16. The kit of claim 15, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

17. The kit of claim 16, wherein the PD-1 inhibitor is selected from nivolumab, pembrolizumab, STI-A1014, pidilzumab, and cemiplimab-rwlc.

18. The kit of claim 16, wherein the PD-L1 inhibitor is selected from avelumab, atezolizumab, durvalumab, and BMS-936559.

19. The kit of claim 16, wherein the CTLA-4 inhibitor is selected from ipilimumab and tremelimumab.

20. The kit of claim 16, wherein the LAG3 inhibitor is GSK2831781.

21. The kit of claim 12, wherein the agent capable of inhibiting PIKfyve activity is ESK981 (13-isobutyl-4-methyl-10-(pyrimidin-2-ylamino)-1,2,4,7,8,13-hexahydro-6H-indazolo[5,4-a]pyrrolo[3,4-c]carbazol-6-one) or a compound structurally similar to ESK981.

22. A method for inhibiting PIKfyve activity in a subject having PIKfyve-expressing cells through administering to the subject a composition comprising a therapeutically effective amount of an agent capable of inhibiting PIKfyve activity (e.g., ESK981 or a compound similar to ESK981).

23. The method of claim 22, further comprising administration to the subject an immune checkpoint inhibitor.

24. The method of claim 23, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

25. The method of claim 24,
wherein the PD-1 inhibitor is selected from nivolumab, pembrolizumab, STI-A1014, pidilzumab, and cemiplimab-rwlc;
wherein the PD-L1 inhibitor is selected from avelumab, atezolizumab, durvalumab, and BMS-936559; wherein the CTLA-4 inhibitor is selected from ipilimumab and tremelimumab;
wherein the LAG3 inhibitor is GSK2831781.

26. A method for inhibiting conversion of phosphatidylinositol 3-phosphate (PI(3)P) to phosphatidylinositol 3,5-bisphosphate (PI(3,5)P₂) in a subject having PIKfyve-expressing cells through administering to the subject a composition comprising a therapeutically effective amount of an agent capable of inhibiting PIKfyve activity (e.g., ESK981 or a compound similar to ESK981).

27. The method of claim 26, further comprising administration to the subject an immune checkpoint inhibitor.

28. The method of claim 27, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

29. The method of claim 28,
wherein the PD-1 inhibitor is selected from nivolumab, pembrolizumab, STI-A1014, pidilzumab, and cemiplimab-rwlc;
wherein the PD-L1 inhibitor is selected from avelumab, atezolizumab, durvalumab, and BMS-936559; wherein the CTLA-4 inhibitor is selected from ipilimumab and tremelimumab;
wherein the LAG3 inhibitor is GSK2831781.

30. A method for inhibiting PIKfyve activity related tumor growth in a subject having PIKfyve-expressing cells (e.g., PIKfyve-expressing cancer cells) through administration to the subject a therapeutically effective amount of an agent capable of inhibiting PIKfyve activity (e.g., ESK981 or a compound similar to ESK981).

31. The method of claim 30, further comprising administration to the subject an immune checkpoint inhibitor.

32. The method of claim 31, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

33. The method of claim 32,
wherein the PD-1 inhibitor is selected from nivolumab, pembrolizumab, STI-A1014, pidilzumab, and cemiplimab-rwlc;
wherein the PD-L1 inhibitor is selected from avelumab, atezolizumab, durvalumab, and BMS-936559; wherein the CTLA-4 inhibitor is selected from ipilimumab and tremelimumab;
wherein the LAG3 inhibitor is GSK2831781.

34. A method for inhibiting PIKfyve activity related autophagic flux in a subject having PIKfyve-expressing cells through administration to the subject a therapeutically effective amount of an agent capable of inhibiting PIKfyve activity (e.g., ESK981 or a compound similar to ESK981).

35. The method of claim 34, further comprising administration to the subject an immune checkpoint inhibitor.

36. The method of claim 35, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

37. The method of claim 36,
wherein the PD-1 inhibitor is selected from nivolumab, pembrolizumab, STI-A1014, pidilzumab, and cemiplimab-rwlc;
wherein the PD-L1 inhibitor is selected from avelumab, atezolizumab, durvalumab, and BMS-936559; wherein the CTLA-4 inhibitor is selected from ipilimumab and tremelimumab;
wherein the LAG3 inhibitor is GSK2831781.

38. A method for activating an anti-tumor immune response in a subject having PIKfyve-expressing cells through administration to the subject a therapeutically effective amount of an agent capable of inhibiting PIKfyve activity (e.g., ESK981 or a compound similar to ESK981) alone or in combination with an immune checkpoint inhibitor as described herein.

39. The method of claim 38, further comprising administration to the subject an immune checkpoint inhibitor.

40. The method of claim 39, wherein the immune checkpoint inhibitor is selected from a PD-1 inhibitor, a PD-L1 inhibitor, a CTLA-4 inhibitor, a LAG3 inhibitor, a TIM3 inhibitor, a cd47 inhibitor, a TIGIT inhibitor, and a B7-H1 inhibitor.

41. The method of claim 40,
wherein the PD-1 inhibitor is selected from nivolumab, pembrolizumab, STI-A1014, pidilzumab, and cemiplimab-rwlc;
wherein the PD-L1 inhibitor is selected from avelumab, atezolizumab, durvalumab, and BMS-936559; wherein the CTLA-4 inhibitor is selected from ipilimumab and tremelimumab;
wherein the LAG3 inhibitor is GSK2831781.

42. A method for inhibiting conversion of phosphatidylinositol 3-phosphate (PI(3)P) to phosphatidylinositol 3,5-bisphosphate (PI(3,5)P₂) in PIKfyve-expressing cells through exposing such cells to compositions comprising an agent capable of inhibiting PIKfyve activity (e.g., ESK981 or a compound similar to ESK981).

43. A method for inhibiting PIKfyve activity in PIKfyve-expressing cells through exposing such cells to compositions comprising an agent capable of inhibiting PIKfyve activity (e.g., ESK981 or a compound similar to ESK981).

44. A method for inhibiting PIKfyve activity related tumor growth in PIKfyve-expressing cells (e.g., PIKfyve-expressing cancer cells) through exposing such cells to compositions comprising an agent capable of inhibiting PIKfyve activity (e.g., ESK981 or a compound similar to ESK981).

45. A method for inhibiting PIKfyve activity related autophagic flux in PIKfyve-expressing cells through exposing such cells to compositions comprising an agent capable of inhibiting PIKfyve activity (e.g., ESK981 or a compound similar to ESK981).

46. A method for activating an anti-tumor immune response in cells having increased PIKfyve activity through exposing such cells to compositions comprising an agent capable of inhibiting PIKfyve activity (e.g., ESK981 or a compound similar to ESK981).


SEQUENCE LISTING (25 sequences)

SEQ ID NO: 1 - DNA, 20 bp, Artificial sequence
  Synthetic
tgcaccaccaactgcttagc

SEQ ID NO: 2 - DNA, 21 bp, Artificial sequence
  Synthetic
ggcatggactgtggtcatgag

SEQ ID NO: 3 - DNA, 23 bp, Artificial sequence
  Synthetic
ggtgagaagagatgtctgaatcc

SEQ ID NO: 4 - DNA, 22 bp, Artificial sequence
  Synthetic
gtccatccttggaagcactgca

SEQ ID NO: 5 - DNA, 22 bp, Artificial sequence
  Synthetic
ctgttcctgcatcagcaccaac

SEQ ID NO: 6 - DNA, 24 bp, Artificial sequence
  Synthetic
tgaactccattcttcagtgtagca

SEQ ID NO: 7 - DNA, 23 bp, Artificial sequence
  Synthetic
ctgagtgatgctgtgtggtcaac

SEQ ID NO: 8 - DNA, 22 bp, Artificial sequence
  Synthetic
caaggactgacacaggcactag

SEQ ID NO: 9 - DNA, 22 bp, Artificial sequence
  Synthetic
actacagcctccattgccacga

SEQ ID NO: 10 - DNA, 22 bp, Artificial sequence
  Synthetic
catcctgtccagacgactgtgt

SEQ ID NO: 11 - DNA, 23 bp, Artificial sequence
  Synthetic
gaagcacctgaataggcaagtcg

SEQ ID NO: 12 - DNA, 23 bp, Artificial sequence
  Synthetic
gagcatccatgaaatctggtcgc

SEQ ID NO: 13 - DNA, 23 bp, Artificial sequence
  Synthetic
catcactgccacccagaagactg

SEQ ID NO: 14 - DNA, 23 bp, Artificial sequence
  Synthetic
atgccagtgagcttcccgttcag

SEQ ID NO: 15 - DNA, 22 bp, Artificial sequence
  Synthetic
atcatccctgcgagcctatcct

SEQ ID NO: 16 - DNA, 24 bp, Artificial sequence
  Synthetic
gaccttttttggctaaacgctttc

SEQ ID NO: 17 - DNA, 24 bp, Artificial sequence
  Synthetic
cctagtgataaggaatgcacgatg

SEQ ID NO: 18 - DNA, 23 bp, Artificial sequence
  Synthetic
ctaggcaggtttgatctccgttc

SEQ ID NO: 19 - DNA, 22 bp, Artificial sequence
  Synthetic
gctccaggatttctcggaagtc

SEQ ID NO: 20 - DNA, 22 bp, Artificial sequence
  Synthetic
atggctactgctgtcaggtcca

SEQ ID NO: 21 - DNA, 19 bp, Artificial sequence
  Synthetic
tggtgtctgcgcctaaatg

SEQ ID NO: 22 - DNA, 20 bp, Artificial sequence
  Synthetic
acgtggggacatatacgtgt

SEQ ID NO: 23 - DNA, 20 bp, Artificial sequence
  Synthetic
acacgtatatgtccccacgt

SEQ ID NO: 24 - DNA, 20 bp, Artificial sequence
  Synthetic
aagagtcagctatttgacgt

SEQ ID NO: 25 - DNA, 20 bp, Artificial sequence
  Synthetic
acgtcaaatagctgactctt